(19) United States
(12) Patent Application Publication
(10) Pub. No.: US 2006/0287696 A1
(43) Pub. Date: Dec. 21, 2006

(54) Heat and light therapy treatment device and method

(76) Inventors: David W. Wright, Littleton, CO (US); William J. Bowers, Highlands Ranch, CO (US); Christopher Andrews, Fort Collins, CO (US); Lee W. Travis, Littleton, CO (US)

Correspondence Address: JOHN R LEY, LLC, 5299 DTC BLVD, SUITE 610, GREENWOOD VILLAGE, CO, 80111, US

(21) Appl. No.: 11/158,305

(22) Filed: Jun. 21, 2005

(51) Int. Cl. A61N 5/06; A61F 7/00; A61F 7/12

(52) U.S. Cl. 607/88; 607/96

(57) ABSTRACT

Heat and light is generated and applied to the skin of a user with multiple arrays of light emitting devices. The amount of heat and light energy delivered from each array is separately controlled relative to a predetermined dose of light energy and a temperature of the skin adjacent to each array. The arrays may be flexibly connected to conform to the contours of a significant area the user's anatomy.

This invention relates to the application of heat and light to living biological tissue for the purpose of therapeutically stimulating the tissue. More particularly, the present invention relates to a new and improved heat and light therapy treatment device and method which offers a convenient, safe and economical way to obtain heat and light therapy.

### BACKGROUND OF THE INVENTION

It is well recognized that the application of artificially-created light to tissue may achieve a therapeutic or healing effect. The application of light to tissue and blood has the effect of influencing the localized release of nitric oxide, thereby stimulating vasodilation. Vasodilation increases blood flow to the affected tissue and brings more of the normal healing effects carried by the blood to the tissue. Light energy has the capability of freeing nitric oxide from the hemoglobin or otherwise releasing nitric oxide from the smooth muscle and vessels. Light energy causes certain photoreactive enzymes to accelerate their functions, thereby enhancing cellular metabolism, blood circulation and nerve function, all of which contribute to healing. In addition to these desirable photochemically-induced effects, the heat resulting from the generation of the artificial light elevates the temperature of the tissue. The increased tissue temperature causes increased blood flow which also contributes to achieving beneficial therapeutic and healing effects of the tissue.

Light and heat have been used to perform and accomplish a wide variety of different types of therapeutic treatments. Coherent and noncoherent light of different wavelengths, intensities and application regimens have been used for specific types of treatments and procedures. Many types of these procedures are destructive in nature, such as surgical procedures where tissue is cut, bleeding tissue is coagulated or tissue is fused together. Other types of these procedures are more homeopathic or natural, such as treatments based on popular concepts of alternative medicine.

The equipment used to generate the light and to apply it in the different types of treatments and procedures can be generally categorized as either very sophisticated, complex and expensive, or relatively simplistic or unsophisticated and therefore not conducive for productive use. The former category of sophisticated, complex and expensive equipment is exemplified by the refined medical equipment that is available for use only by skilled professional medical technicians, such as laser devices. The use of this type of sophisticated equipment is generally limited to medical facilities, such as hospitals and clinics. The latter category of unsophisticated and simplistic equipment may generally be considered a consumer product which is oriented toward use by an ordinary individual. This type of equipment is usually straightforward and simple to the point where its simplicity interferes with its ability to achieve a positive result. The unsophisticated type of equipment is relatively inexpensive, because the market for such equipment is an average consumer who is unwilling to spend a significant amount of money for equipment that may have marginal or questionable value. Consequently, the relatively inexpensive equipment has not had a reputation for achieving significant therapeutic and healing results, primarily because of the manner in which it has been designed and constructed.

### SUMMARY OF THE INVENTION

The present invention offers consumers a very well functioning and therefore effective heat and light therapy treatment device which provides exceptional functionality in delivering heat and light energy in an effective and economical manner for therapeutic and healing purposes. The present invention also facilitates convenient, straightforward and effective use of the heat and light therapy treatment device and methodology, thereby making it easier for consumers to achieve positive therapeutic and healing results from the use of the equipment.

More specifically, the therapy device of the present invention uses individual heat and light therapy modules which are flexibly connected together to permit the therapy modules to adapt comfortably to, and cover, the tissue over the contours of the user's body. The heat and light energy from each therapy module is individually controlled at each location where the therapy module contacts the skin of the user. The individual control of the light from each therapy module permits different tissue types, such as thin skin covering bony prominences and thick tissue covering more massive physiology, to obtain improved heat and light therapy without reaching increased temperatures where diminishing benefits occur. Consequently, the heat and light therapy is more uniform and effectively delivered according to the type of tissue. Flexibly linking multiple therapy modules allows the heat and light therapy to be applied over relatively large areas of tissue. The size and shape of the therapy modules make them convenient for use, such as by permitting them to be worn under clothing or held in the desired position for the treatment by easily connected and adjusted straps. The internal functionality of the device, as well as its external functionality in delivering the heat and light to the tissue, is monitored and controlled to prevent deviations from expected operation. The structural organization and construction of the therapy device allows it to be manufactured at a relatively reasonable price that is affordable by those individuals who wish to use the device for homepathic or natural reasons. The relatively high level of functionality the device makes advantageous for medically prescribed treatments. Similar and related benefits, advantages and improvements are also available from the methodology of the present invention.

These and other features are achieved by a therapy device for generating heat and light and applying the generated heat and light to the skin of a user. The therapy device includes a plurality of therapy modules. Each of the therapy modules includes an array of a plurality of light emitting devices which generate heat when emitting light. Each module has a housing with a window through which passes the heat and light generated by the light emitting devices. Each therapy module further includes electronic circuitry located within the housing with which to control the application of electrical energy to the light emitting devices. At least one flexible coupler connects adjoining pairs of therapy modules into a single configuration formed by the plurality of connected therapy modules. Electrical conductors are included in each flexible coupler to conduct electrical power between the electronic circuitry located within the housings of the adjoining pairs of therapy modules. A control module is connected by a cable to one of the plurality of therapy modules. The control module includes circuit components which supply electrical power through the cable to the electronic circuitry located in the housing of the one therapy module. The conductors of the flexible couplers distribute the electrical power from the one therapy module to the other therapy modules in the configuration.

Preferred features of the therapy device include some or all of the following. A temperature sensor is located within each housing. The temperature sensor is in thermal contact with the skin of the user when the window of the housing is placed in contact with the skin of the user. The electronic circuitry of each therapy module controls the electrical energy applied to the light emitting devices to control the temperature of the skin contacted by each therapy module by controlling the light and heat emitted from the light emitting devices. The window includes a protrusion to physically contact the skin of the user and a stud extending into the housing from the window on the opposite side of the protrusion. The protrusion is directly thermally connected to the temperature sensor, thereby establishing a thermally conductive path directly from the skin of the user to the temperature sensor. The circuit components of the control module include a controller for timing the duration of electrical power supplied to the therapy modules. The controller initiates the supply of electrical power at the commencement of a therapy treatment and terminates the supply of electrical power at the end of the therapy treatment. A clock signal having a predetermined frequency is used for timing the duration of the therapy treatment. Deviations from the predetermined frequency of the clock signal are monitored and the supply of electrical power is terminated upon detecting a substantial deviation. The controller measures the time between the termination of a preceding therapy treatment and the commencement of a subsequent therapy treatment and adds time to the duration of electrical power supplied for the therapy treatment when the measured time between the termination of the preceding therapy treatment and the commencement of the subsequent therapy treatment indicates that the light emitting devices will emit light of reduced intensity due to residual temperature of the light emitting devices resulting from the preceding therapy treatment. Low-power and high-power control switches may be selectively activated to create a relatively longer time duration for a low-power therapy treatment and a relatively shorter time duration for a high-power therapy treatment. The electrical energy applied to the light emitting devices is controlled to increase the amount of light and heat emitted when high-power therapy treatment is selected, and the electrical energy is controlled to decrease the amount of light and heat emitted when the low-power therapy treatment is selected. The plurality of therapy modules are connected in the configuration by the use of a flexible circuit having a substantially flat continuous flexible insulating substrate upon which traces are formed as the electrical conductors, and flexible plastic material is molded over and surrounds the flexible circuit to mechanically connect the housings of the adjoining therapy modules. The electronic circuitry within the housing of each therapy module includes a first temperature sensor in direct thermal contact with the skin of the user and a second temperature sensor within the housing of the therapy module. The electrical energy applied to the light emitting devices during low-power therapy is regulated in response to the temperatures sensed by both the first and second temperature sensors, but is regulated in response to the temperature sensed by the first sensor in high-power therapy. The plurality of therapy modules in the configuration may form a linear row with terminal couplers connected at the ends of the row. A strap is connected to the terminal couplers to hold the row of therapy modules on the user. The plurality of therapy modules may also form a two-dimensional configuration. The control module includes a body within which its circuit components are located, and an attachment clip is connected to the body for mechanically connecting the control module to an object worn by the user, such as a belt or pocket.

Other features of the invention are achieved by a method for generating heat and light and applying the generated heat and light to the skin of a user. The method includes organizing a plurality of light emitting devices in an array, generating heat and light by supplying electrical energy to each light emitting device in the array, positioning a plurality of separate arrays to deliver heat and light to substantially adjoining but separate areas of the user's skin, and separately controlling the electrical energy applied to the light emitting devices of each array to regulate the temperature of the skin at each separate area independently of the temperature of the skin at the adjoining separate areas.

Preferred features of the method include the following. A plurality of the arrays are flexibly connected together, electrical energy is applied to the light emitting devices of each array through at least one of the flexible couplings to each array, electrical power is conducted to the electronic circuitry of the adjoining pairs of therapy modules, and electrical power is supplied through a cable to one array and distributed from the one array through the flexible couplings to the other arrays. The plurality of arrays are flexibly connected together with a flexible circuit which has a substantially flat continuous flexible insulating substrate upon which traces are formed as the electrical conductors by which to deliver electrical power and a control signal to the plurality of arrays. Flexible plastic material is molded over and surrounds the flexible circuit between the separate and flexibly coupled arrays. The temperature of the skin of the user is sensed through direct thermal contact. The duration of electrical power supplied to the arrays is timed to establish the duration of a therapy treatment. A clock signal is delivered at a predetermined frequency by which to time the duration of electrical power supplied, the clock signal is monitored for deviations from the predetermined frequency, and the supply of electrical power is terminated upon detecting that the frequency of the clock signal has deviated significantly from the predetermined frequency. The time between the termination of a preceding therapy treatment and the commencement of a subsequent therapy treatment is measured, and time is added to the duration of electrical power supplied if the measured time between the termination of the preceding therapy treatment and the commencement of the subsequent therapy treatment indicates that the light emitting devices will emit light of reduced intensity due to their residual temperature from use during the preceding therapy treatment. Either a low-power therapy treatment having a relatively longer time duration or a high-power therapy treatment having a relatively shorter time duration is selected. The electrical energy applied to the light emitting devices of each array is separately controlled to modulate the amount of light and heat emitted in a specific time from each array in relation to a predetermined anticipated dose or amount of light energy and/or a predetermined anticipated temperature of the user's skin. The temperature of the user's skin is directly thermally conducted to a first temperature sensor, and the temperature generally surrounding the array is sensed with a second temperature sensor associated with each array. The electrical energy applied to the light emitting devices of each array is controlled in response to the temperatures sensed by the first and second temperature sensors in relation to the selected high-power and low-power therapy. The plurality of arrays may be formed into a two-dimensional configuration by a plurality of laterally adjacent rows of arrays.

A more complete appreciation of the scope of the present invention and the manner in which it achieves the above-noted and other improvements can be obtained by reference to the following detailed description of presently preferred embodiments taken in connection with the accompanying drawings, which are briefly summarized below, and by reference to the appended claims.

### BRIEF DESCRIPTION OF THE DRAWINGS

FIG. 1 is a perspective view a heat and light therapy device embodying the present invention.

FIG. 2 is a perspective view of a heat and light applicator portion of the device shown in FIG. 1, formed by a linear row of heat and light therapy modules, illustrated in an inverted relationship compared to FIG. 1.

FIG. 3 is an exploded perspective view of the linear row of heat and light therapy modules, illustrated in the relationship shown in FIG. 1.

FIG. 4 is an enlarged view of a portion of FIG. 3, illustrating components of one heat and light therapy module and illustrating broken away portions of couplers which connect the therapy modules in the linear row.

FIG. 5 is an enlarged vertical cross-sectional view of one therapy module taken substantially in the plane of line 5-5 in FIG. 1.

FIG. 6 is an enlarged vertical cross-sectional view of one coupler and portions of the therapy modules to which it connects taken substantially in the plane line of 6-6 in FIG. 1.

FIG. 7 is an enlarged perspective view of one coupler which connects adjacent therapy modules as shown in FIG. 1.

FIG. 8 is a top plan view of the coupler shown in FIG. 7.

FIG. 9 is a vertical cross-sectional view of the coupler taken substantially in the planes of lines 9-9 in FIGS. 7 and 8.

FIG. 10 is an enlarged partial perspective view of a cable end terminal coupler, a flexible circuit and a cable, as well as a perspective view of a hook clasp of the therapy device shown in FIG. 1.

FIG. 11 is an enlarged perspective view of the cable end terminal coupler and the hook clasp shown in FIG. 10.

FIG. 12 is an enlarged perspective view of a row end terminal coupler and hook clasp of the therapy device shown in FIG. 1.

FIG. 13 is a vertical cross-sectional view of the cable end terminal coupler and the hook clasp shown in FIG. 11.

FIG. 14 is a vertical cross-sectional view similar to FIG. 13, showing the hook clasp connected to the cable end terminal coupler.

FIG. 15 is a vertical cross-sectional view of the cable end terminal coupler shown in FIG. 13, to which a strap has been directly connected.

FIG. 16 is an enlarged side elevational view of the control module shown in FIG. 1.

FIG. 17 is an exploded perspective view of the control module shown in FIG. 16.

FIG. 18 is a circuit diagram of electronic components of each heat and light therapy module shown in FIG. 1.

FIGS. 19A and 19B are waveform diagrams having a common time reference which illustrate certain signals present in the circuit shown in FIG. 18.

FIGS. 20A and 20 B are waveform diagrams having a common time reference which illustrate certain signals present in the circuit shown in FIG. 18.

FIG. 21 is a circuit diagram of certain electronic components of the control module shown in FIG. 1.

FIGS. 22A and 22B are waveform diagrams having a common time reference which illustrate certain signals present in the circuit shown in FIG. 21.

FIG. 23 is a graph of light intensity emitted by a light emitting diode (LED) of the therapy module shown in FIG. 1 in relation to the time during which that LED has been energized.

FIG. 24 is a graph of light intensity emitted by a light emitting diode of the therapy module shown in FIG. 1 in relation to its temperature.

FIG. 25 is a flowchart illustrating aspects of a process flow executed by a microprocessor of the control module shown in FIG. 21.

FIG. 26 is a perspective view of a heat and light applicator formed by a two-dimensional configuration of heat and light therapy modules.

### DETAILED DESCRIPTION

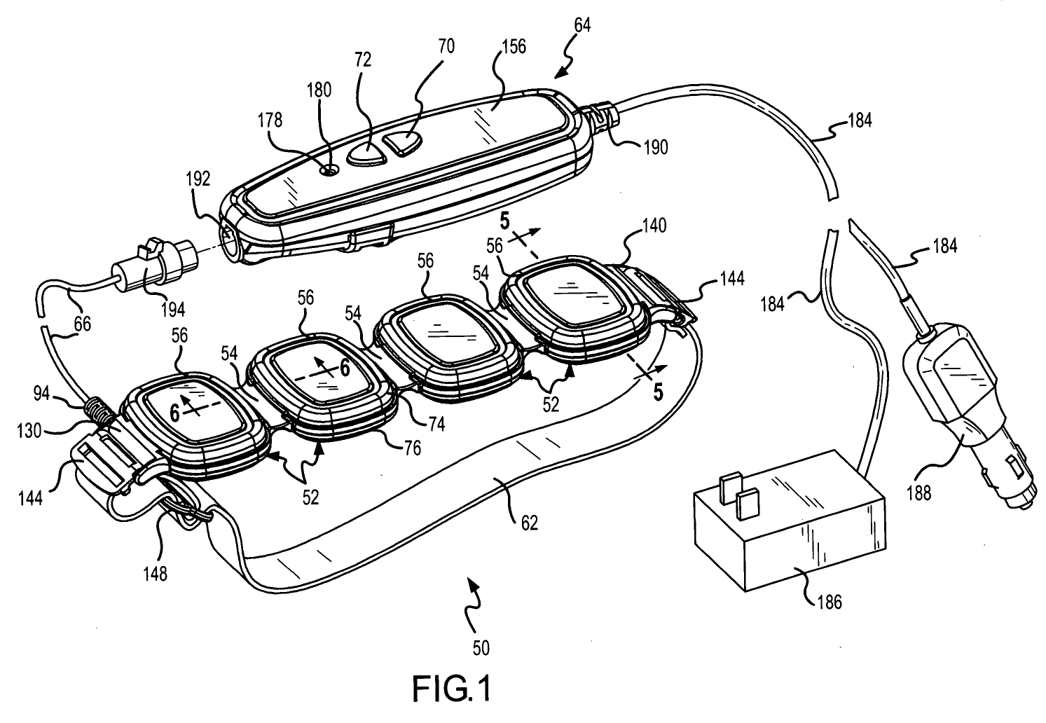

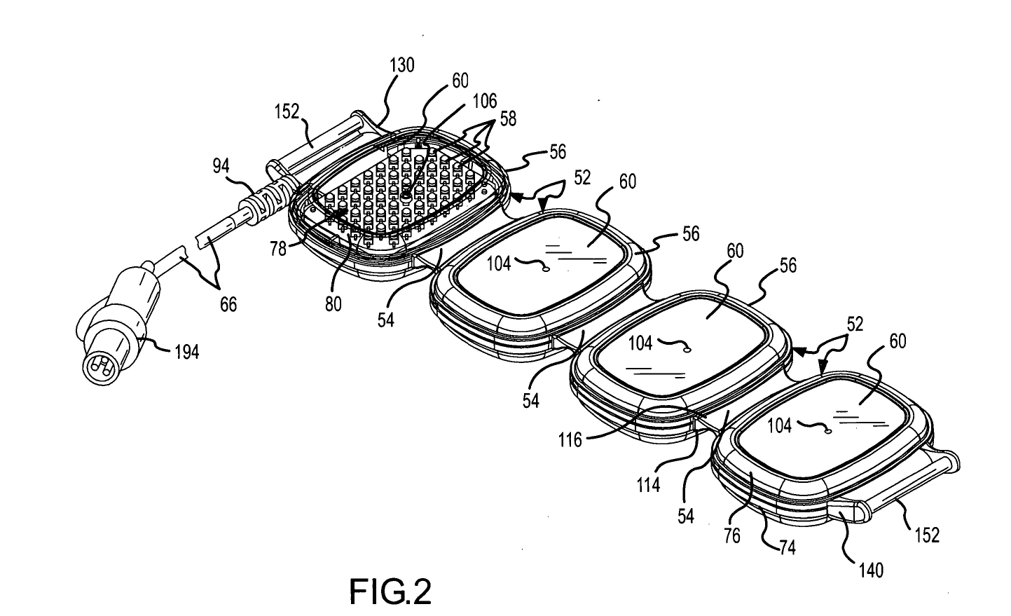

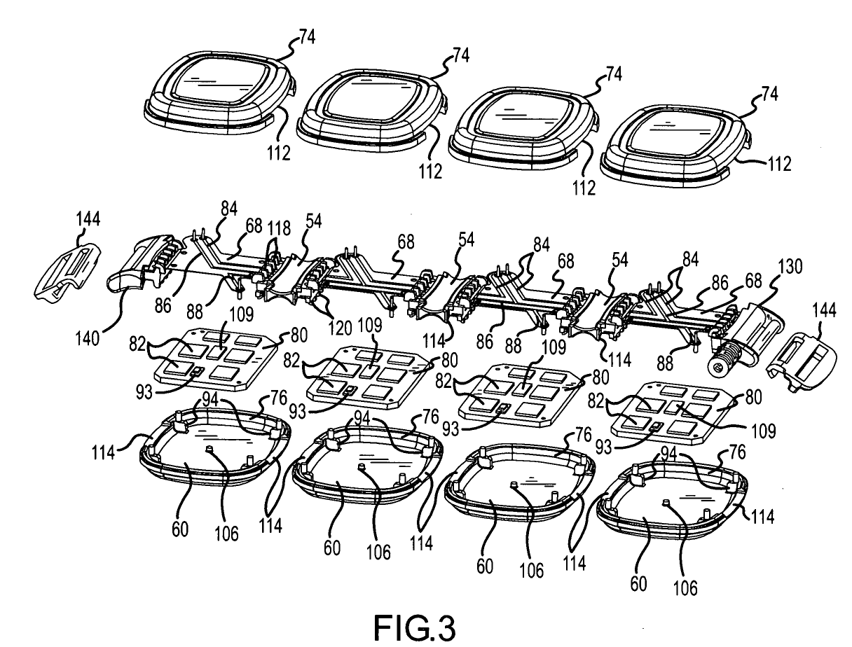

A therapy device 50 for applying therapeutic heat and light the skin of a user is shown in FIG. 1. The therapy device 50 includes at least one, and preferably a plurality of, heat and light applying therapy modules 52 which are connected in the configuration of a linear row by flexible couplers 54, as also shown in FIGS. 2 and 3. Each therapy module 52 includes a housing 56 within which there is located a plurality of LEDs (light emitting diodes or devices) 58 arranged in a two-dimensional array. A clear, transparent or light transmissive window 60 in the housing 56 permits the light from the LEDs 58 to impinge on the skin of the user when the windows 60 of the therapy modules 52 are placed in contact with, or facing, the skin of the user at an area where a therapeutic effect is desired. A connection strap 62 extends between opposite ends of the row of therapy modules 52 to hold them in conforming contact with an area of skin and tissue where the therapy is applied.

The application of light to the tissue achieves a therapeutic or healing effect as a result of the light interacting photochemically with the tissue, the blood, and the various components of the blood and tissue. In addition, the heat resulting from the transmitted light and from the generation of the light by the LEDs 58 physically elevates the temperature of the tissue, which also achieves and contributes to the therapeutic or healing effect.

Each therapy module 52 also includes an individual capability to respond to the temperature of the skin and tissue of the user adjacent to its window 60. Each therapy module 52 individually adjusts the amount of heat and light energy emitted from its array of LEDs 58, separately from the amount of heat and light supplied by the other therapy modules 52 in the linear row. The individual temperature control from each therapy module 52 has the benefit of limiting the heat and light to those areas which are covered by relatively thin skin and tissue over bones, such as at an elbow joint for example, while applying more extensive heat and light to the skin over more massive tissues, such as over a large muscle, for example. In this way, the row of therapy modules 52 may be used in contact with different types and thicknesses of tissue without excessively or inadequately heating areas of tissue and skin. Individualized heat and light therapy is applied to each area of skin and tissue contacted by each therapy module 52.

The therapy device 50 also includes a control module 64, shown in FIG. 1. The control module 64 supplies electrical energy to the therapy modules 52 and also exercises certain control functions with respect to the overall operation of, and treatment delivered by, the therapy device 50. A cable 66 supplies electrical power and control signals to the therapy modules 52. A flexible circuit 68 (FIG. 3) is molded into the couplers 54 and is electrically connected to the cable 66. The flexible circuit 68 extends along the row of therapy modules 52 and distributes the electrical power and control signals from the control module 64 and the cable 66 to each of the therapy modules 52.

The control module 64 controls the overall time duration of the treatment by terminating the treatment after the energy has been applied to the tissue for a predetermined treatment time. The control module 64 also permits the user to select either a low-power therapy treatment or a high-power therapy treatment, by depressing a low-power selector button 70 or a high-power selector button 72, respectively. A high-power treatment delivers a greater amount of heat and light energy for a relatively shorter amount of time, while a selected low-power treatment delivers a lesser amount of heat and light energy for a relatively longer amount of time. The same total amount of light energy is delivered, but only the treatment time varies. Alternatively, different quantities of energy may be delivered in the high-power and low-power therapy treatments. The control module 64 communicates control signals indicative of the selected high- and low-power treatments to the therapy modules 52. The control module 64 times the duration of the selected treatment and terminates the electrical power delivered to the therapy modules 52 at the end of the selected treatment. The control module 64 also performs certain oversight functions to prevent an overdose of heat and light energy, which can reverse or limit the therapeutic effect, if an internal timing malfunction should occur.

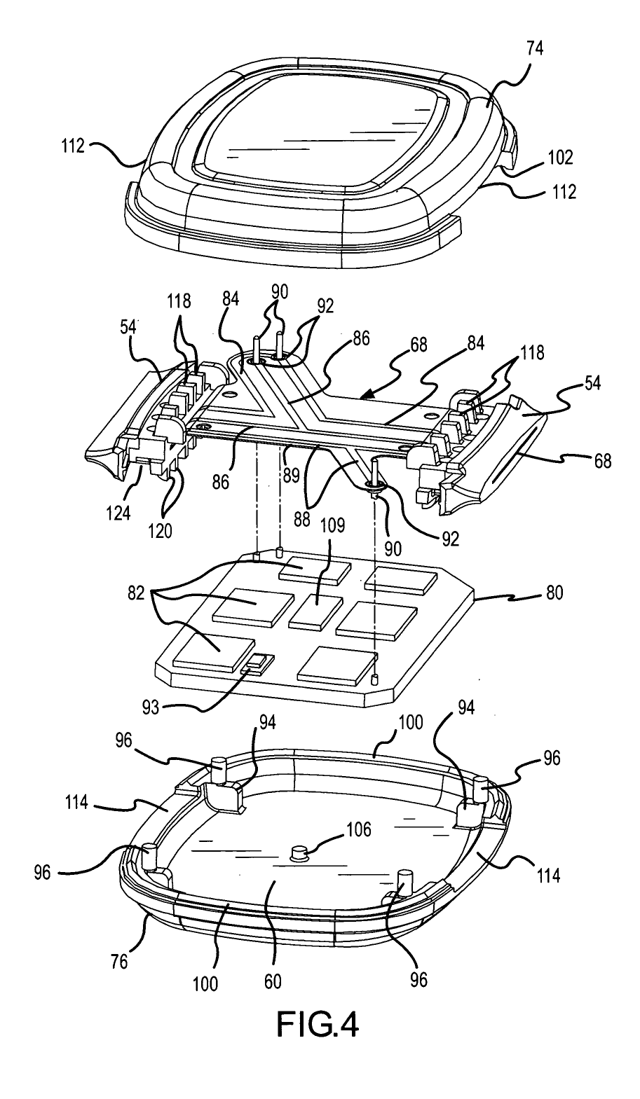

Each therapy module 52 has an upper shell 74 and a lower shell 76 which are joined together to form the housing 56, as shown in FIGS. 3 and 4. The lower shell 76 includes the window 60 which allows light from the array of LEDs 58 inside of the housing 56 to pass to the exterior of the housing 56 where the light is applied to the user. The window 60 is preferably made from polycarbonate, which is strong and durable as well as being transparent to light radiation. The polycarbonate is also a thermal conductor of the heat energy from the LEDs 58 to the skin and tissue of the user and from the skin and tissue into the housing. The LEDs 58 are generally arranged in a two-dimensional array 78 (FIG. 2) on a circuit board 80. The circuit board 80 is located in the housing 56 of each therapy module 52. The LEDs 58 are located on the side of the circuit board 80 which faces and is adjacent to the window 60.

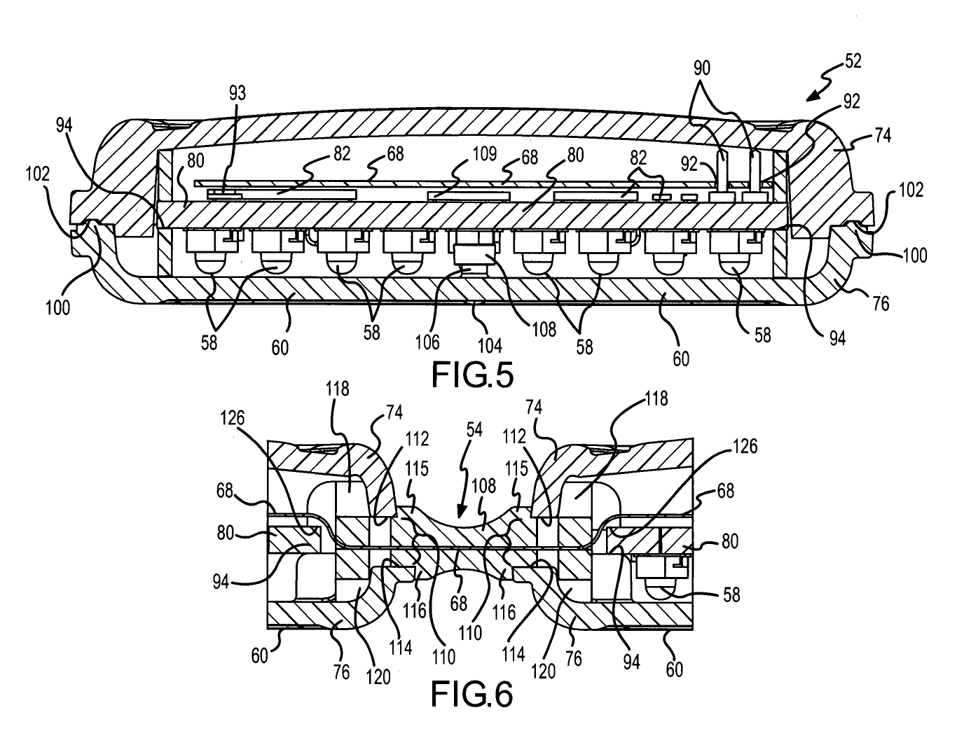

The array 78 has multiple rows of LEDs 58 with multiple LEDs in each row, as shown in FIG. 2. The LEDs 58 of each row are slightly transversely offset from the LEDs 58 of the adjacent row to establish a relatively tight packing or positioning of LEDs 58 in the array 78. The circuit boards 80 are conventional printed circuit boards with circuit traces to conduct electrical signals between electrical components 82, as shown in FIGS. 3 and 4. For the most part, the electrical components 82 are mounted on the opposite side of the circuit board from the LEDs 58, which allows the LEDs 58 to be arranged in the array 78 without interference from the electrical components 82, as is also shown in FIG. 5.

The circuit board 80 is connected to circuit traces 84, 86 and 88 of the flexible circuit 68, through which the circuit board 80 receives electrical power and control signals from the control module 64. The flexible circuit 68 is of the conventional construction formed by a substantially flat continuous flexible insulating substrate 89 upon which the traces 84, 86 and 88 are formed as electrical conductors. The flexible circuit 68 extends from the ends of the couplers 54 into the housing 56 of each therapy module 52 and over the circuit board 80, as shown in FIGS. 3-5. Solder pins 90 extend upward from conductors on the circuit board 80 through solder holes 92 of the circuit traces 84, 86 and 88 in the flexible circuit 68. The solder pins 90 are soldered to the circuit traces 84, 86 and 88 at the solder holes 92 to electrically connect the flexible circuit 68 to each circuit board 80. The circuit traces 84, 86 and 88 connect to each circuit board 80 on the same side of the circuit board 80 as the electrical components 82. Connecting the flexible circuit 68 to the side of the circuit board 80 opposite of the LEDs 58 allows the light to pass through the window 60.

An LED 93 is connected to the circuit board 80 on the same side as the components 82, which is on the opposite side from the LEDs 58. The LED 93 emits light when heat and light energy is delivered by the therapy module 52. The light from LED 93 passes through the upper shell, which is translucent, to indicate to the user that heat and light energy is being applied to the tissue. The intensity of the light transmitted by the energy delivery indicator LED 93 represents the intensity of the energy conducted by the LEDs 58 to the tissue.

The circuit board 80 with the array 78 of LEDs 58 and the electrical components 82, a portion of the flexible circuit 68, and the ends of the couplers 54, are all trapped between the upper shell 74 and the lower shell 76. The lower shell 76 has shelves 94 which support the circuit board 80 and space the array 78 of LEDs 58 relative to the window 60.

The lower shell 76, as shown in FIG. 4, includes attachment posts 96 which are inserted into correspondingly aligned attachment holes (not shown) in the upper shell 74 to secure the upper and lower shells 74 and 76 together and form the housing 56. The posts 96 and holes align the upper and lower shells 74 and 76 so that a raised rim 100 on the lower shell 76 is positioned in a recess 102 in the upper shell 74 (FIGS. 4 and 5) when the shells 74 and 76 are assembled. To assemble each therapy module 52, the raised rim 100 and the recess 102 are secured together by an adhesive, or thermal or ultrasonic welding, to connect the upper shell 74 to the lower shell 76.

The exterior of the window 60 in the lower shell 76 includes a slight protrusion 104 (FIGS. 2 and 5) which contacts the tissue of the user when the window 60 of the therapy module 52 is placed in contact with the tissue. The protrusion 104 extends outward from the surface of the window 60 and is made from the same material as the window 60. The protrusion 104 is positioned on the window 60 near the center of the array 78 of LEDs 58. The protrusion 104 ensures good physical contact with the skin of the user when the window 60 is placed in contact with the skin.

The protrusion 104 thermally assumes the temperature of the skin at the position where the window 60 of each therapy module 52 contacts the user. A raised portion or stud 106 is on the opposite side of the window 60 from the protrusion 104 and is in thermal contact with the protrusion 104. The stud 106 thermally contacts a main thermistor 108, which is one of the components 82 that is mounted on the circuit board 80. The main thermistor 108 responds to the temperature of the stud 106 as influenced by the skin temperature and the temperature within the housing 56. The main thermistor 108 creates a signal related to the temperature sensed, and that temperature reference signal is instrumental in causing the other components 82 on the circuit board 80 to control and regulate the amount of energy transmitted by the LEDs 58. Controlling the amount of heat energy transmitted by the LEDs 58 elevates the skin and tissue to a therapeutic temperature while also delivering enough light energy to achieve therapeutic effects.

An auxiliary thermistor 109 is connected to the circuit board 80 on the opposite side from which the main thermistor 108 is connected. The auxiliary thermistor 109 creates a signal related to the temperature within the housing 56 of each therapy module 52. The signal from the auxiliary thermistor 109 is used in low-power therapy treatments, in combination with the signal from the main thermistor 108, to establish and regulate the heat and light energy delivered during low-power therapy treatment.

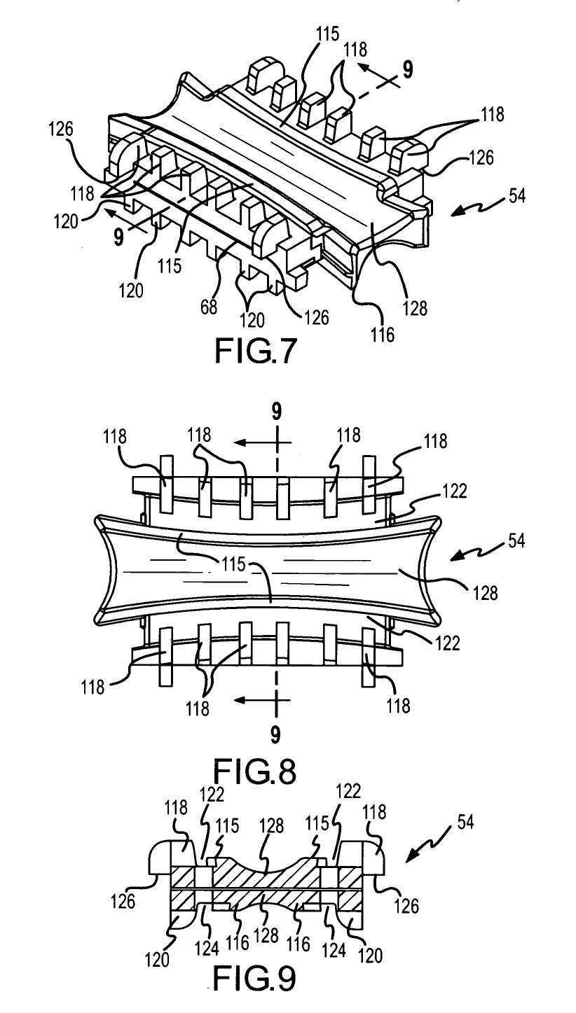

Each coupler 54 is preferably formed from resilient electrically-insulating plastic material, such as silicone, which has been molded over and around the flexible circuit 68, as shown in FIGS. 6-9. The overmolding causes each coupler 54 to adhere to the flexible circuit 68. Consequently, each coupler 54 protects the portion of the flexible circuit 68 which extends between the therapy modules 52. The couplers 54 have a resilient characteristic, which when combined with the flexibility of the flexible circuit 68, allows the therapy modules 52 to bend relative to one another when conforming to the contours of the user's anatomy. On the other hand, the couplers 54 are firmly connected to the housings 56 of the therapy modules 52, to prevent stretching of the flexible circuit 62 between the adjacent therapy modules 52.

Ends of the couplers 54 fit in slots 110 in the housings 56. The slots 110 are each formed by a recess 112 in the upper shell 74 and a recess 114 in the lower shell 76, as shown in FIGS. 3, 4 and 6. The ends of the couplers 54 are retained or trapped in the slots 110 when the shells 74 and 76 are secured together to form the housing 56. As shown in FIGS. 6-9, each end of each coupler 54 has exterior upper and lower raised flanges 115 and 116 which contact the exterior of the upper and lower shells 74 and 76 of the assembled housing 56, respectively. Each end of each coupler 54 has internal fingers 118 that extend upward and engage the interior of each upper shell 74, and internal fingers 120 which extend downward and engage the interior of each lower shell 76, adjacent to the recesses 112 and 114, respectively. The upper shell recess 112 fits into an upper channel 122 which extends between the upper flanges 115 and the upper fingers 118. The lower shell recess 114 fits into a lower channel 124 between the lower flange 116 and the lower fingers 120. The channels 122 and 124 interact with the shells 74 and 76 at the recesses 112 and 114 to retain the therapy modules 52 relative to one another. By engaging the inside surfaces of the upper and lower shell 74 and 76, the fingers 118 and 120 prevent the ends of the couplers 54 from being pulled out of the slots 110. The exterior flanges 114 and 116 prevent the couplers 54 and the flexible circuit 68 from being pushed into the interiors of the therapy modules 52.

Lower downward-facing edges 126 on the upper fingers 118 contact the upper surface of the circuit board 80 within the interior of each therapy module 52, as shown in FIG. 6. The resiliency of the material from which the upper fingers 118 is made applies a resilient downward force on the upper surface of the circuit board 80 to hold the circuit board 80 in position against the shelves 94. In this manner, trapping the ends of the couplers 54 within the interior of the housing 56 also retains the circuit board with its LEDs 58 and electrical components 82 in a fixed position within each therapy module 52.

A reduced thickness web portion 128 of the couplers 54 extends between the exterior flanges 115 and 116 of the couplers 54. The web portion 128 surrounds the flexible circuit 68 and permits the bending of the couplers 54 between the therapy modules 52, while still protecting and supporting the flexible circuit 68.

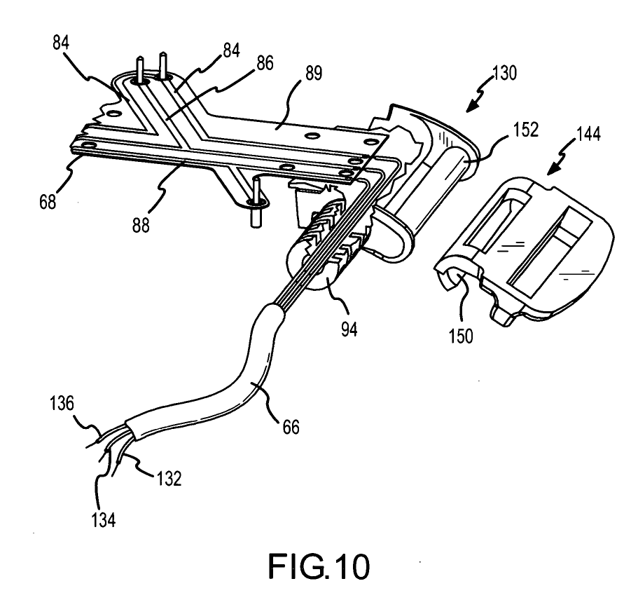

As shown in FIG. 10, a cable end terminal coupler 130 permits an electrical and mechanical connection between the traces 84, 86 and 88 of the flexible circuit 68 and electrical conductors 132, 134 and 136, respectively. The electrical conductors 132, 134 and 136 extend within the cable 66 from the control module 64 and are soldered or otherwise electrically and mechanically connected to the traces 84, 86 and 88 within the terminal conductor. The cable end terminal coupler 130 is molded over and around the cable 66 and over the flexible circuit 68 after the conductors 132, 134 and 136 have been electrically connected to the traces 84, 86 and 88, respectively. The overmolding assists in maintaining a mechanical and electrical connection between the conductors 132, 134 and 136 and the traces 84, 86 and 88. A strain relief 94 holds the cable 66 within the cable end terminal coupler 130.

A row end terminal coupler 140, shown in FIG. 12, is connected to the opposite end of the row of therapy modules 52 opposite from the cable end terminal coupler 130. The row end terminal coupler 140 is formed by overmolding it around the end of the flexible circuit 68 which is opposite from the end of the flexible circuit 68 to which the cable end terminal coupler 130 is overmolded. The cable end terminal coupler 130 and the row end terminal coupler 140 attach to their adjacent therapy modules 52 in a manner similar to the attachment of the intermediate flexible couplers 54 to their adjacent therapy modules 52.

The cable and row end terminal couplers 130 and 140 include upper fingers 118 and lower fingers 120 of the same characteristics as those of the couplers 54. An upper channel 122 and a lower channel 124 are formed between the upper and lower fingers 118 and 120 and a main portion 138 of the cable end terminal coupler 130 and a main portion 142 of the row end terminal coupler 140. The channels 122 and 124 have the same characteristics as those of the couplers 54. The recesses 112 and 114 of the upper and lower shells 74 and 76 fit within the upper and lower channels 122 and 124 when the housing 56 is assembled by connecting the upper and lower shells 74 and 76. Connecting the upper and lower shells 74 and 76 holds the cable and row end terminal couplers 130 and 140 to their adjoining therapy modules 52. Outward or inward movement of the terminal couplers 130 and 140 relative to their adjoining therapy modules is prevented in the same manner as outward and inward movement of the couplers 54 between their adjacent therapy modules 52 is prevented. Lower downward-facing edges 126 on the upper fingers 118 of the cable and row end terminal coupler 130 and 140 contact the upper surfaces of the circuit boards 80 within the interiors of the adjacent therapy modules 52 to hold the circuit board 80 in position in the end therapy modules 52 in the same manner that the couplers 54 hold the circuit boards 80 in position within the intermediate therapy modules 52.

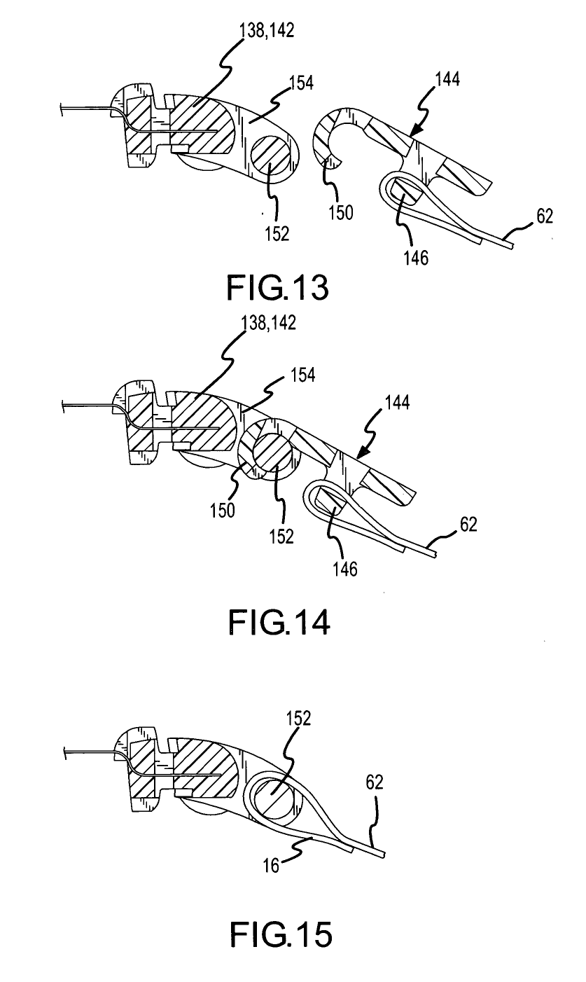

The connection strap 62 connects to the terminal couplers 130 and 140 on opposite ends of the row of therapy modules 52, as shown in FIGS. 1 and 13-15. Hook clasps 144 are attached to opposite ends of the connection strap 62. The hook clasps 144 connect the strap 62 to the terminal couplers 130 and 140. The connection strap 62 is connected to each hook clasp 144 by extending an end of the strap 62 around a bridge 146 of each hook clasp 144 and attaching the end back to the main portion of the strap 62 on the opposite side of the bridge 146, as shown in FIGS. 13 and 14. Alternatively, the end of the strap 62 can be attached to a buckle-type adjustor 148 on the main portion of the strap 62, after the end has been passed around the bridge 146, as shown in FIG. 1.

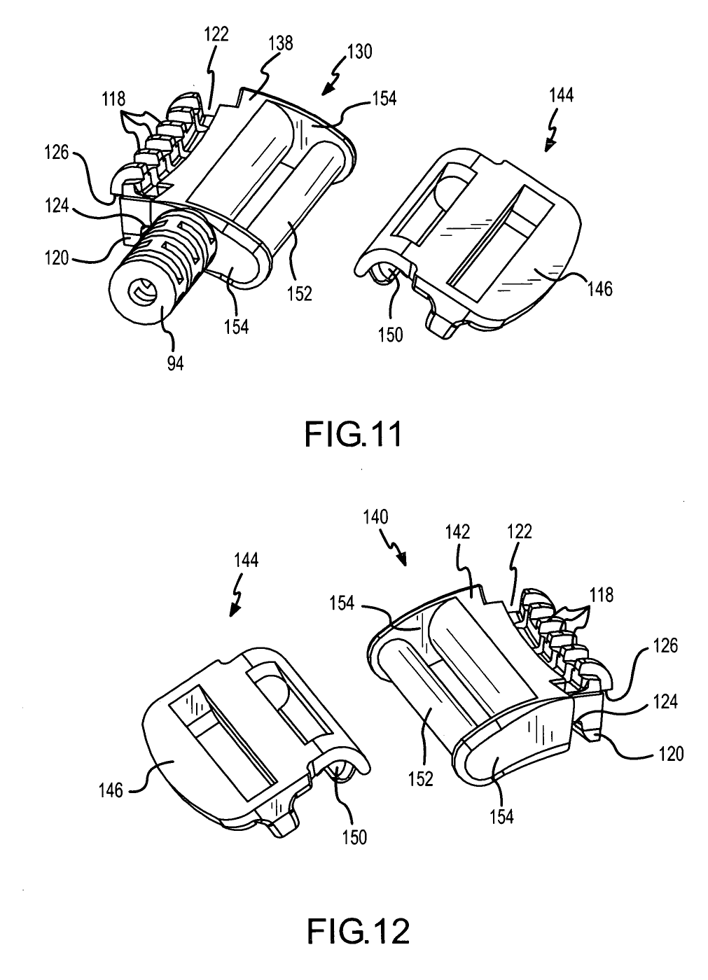

The hook clasps 144 attach to the terminal couplers 130 and 140 by connecting a hook portion 150 of each clasp 144 around a connection shaft 152 of each terminal coupler 130 and 140, as shown in FIGS. 11-14. Parallel arms 154 extend outward from the main portions 138 and 142 of the terminal couplers 130 and 140, and the connection shaft 152 extends between the arms 154 to accept the hook portion 150 of each clasp 144. The hook portion 150 of each hook clasp 144 is inserted into the space between the shaft 152 and the main portion 138 and 142. The hook portion 150 is then pulled into place around the shaft 152 where the connection is maintained by tension in the strap 62. Both the connection shaft 152 and the hook portion 150 are cylindrically shaped. The hook portion 150 extends slightly more than 180 degrees around the cylindrical shaft 152, as shown in FIG. 14. The hook portion 152 deflects slightly so that it will expand and fit over the shaft 152 and then resume its original shape while surrounding the shaft 152. In this manner, each clasp 144 is retained to each terminal coupler 130 and 140. However, the retention force permits each hook clasp 144 to pivot slightly around the connection shafts 152 of the terminal couplers 130 and 140. Each hook clasp 144 can be disconnected from the terminal coupler 130 or 140 by deflecting the hook portion 150 and separating the hook clasp 144 from the connection shaft 152.

The strap 62 may also be directly connected to either of the terminal couplers 130 or 140 without the use of the hook clasps 144, as shown in FIG. 15. A direct connection is achieved by looping the strap 62 around the connection shaft 152, and holding the free end of each strap to the adjoining portion of the strap 62 with a conventional buckle-type adjustor, such as that shown at 148 in FIG. 1. One end of the strap 62 could be permanently attached to one shaft 152 on one of the terminal couplers 130 or 140, so long as the other end of the strap remains free and adjustable with respect to the other shaft 152 on the other one of the terminal couplers 130 or 140.

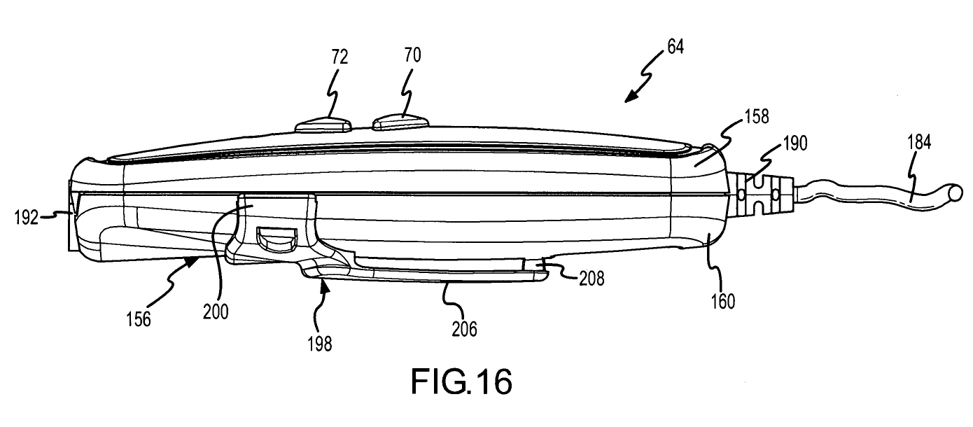

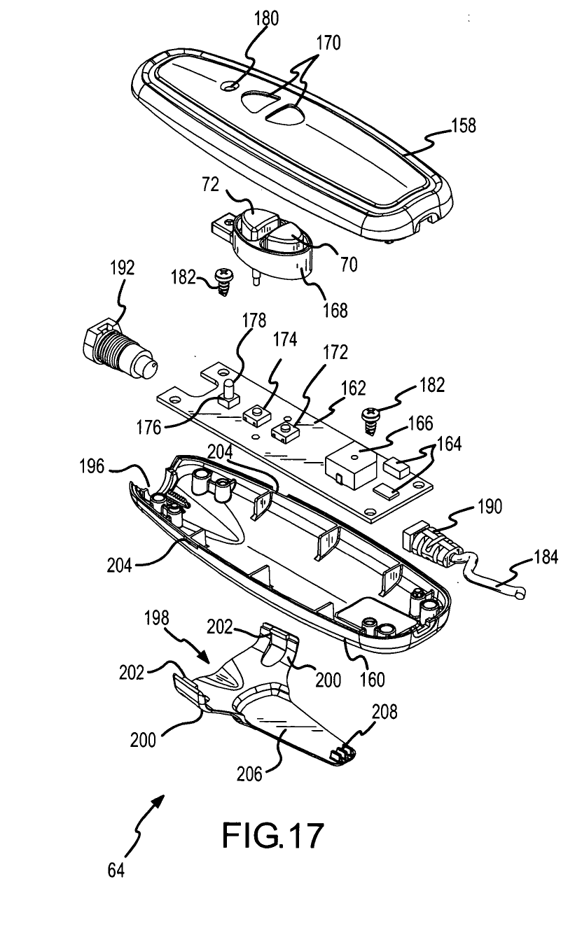

Details of the control module 64 are shown in FIGS. 16 and 17. The control module 64 supplies electrical power to the components 82 of the circuit board 80 in each of the therapy modules 52 by conducting supply voltage on the electrical conductor 134, which is connected to the trace 86 of the flexible circuit 68, and by providing reference potential on the electrical conductor 136 which is connected to the trace 88 of the flexible circuit 68 (FIGS. 3, 4 and 10). In addition, the control module 64 conducts control signals on the electrical conductor 132 which is connected to the trace 84 of the flexible circuit 68 (FIGS. 3, 4 and 10). The therapy control signal on the trace 84 is interpreted by the components 82 of the circuit board 80 in each therapy module 52 to control the delivery of either the high or low-power therapy treatments, as selected by the user depressing the selector buttons 72 or 70, respectively.

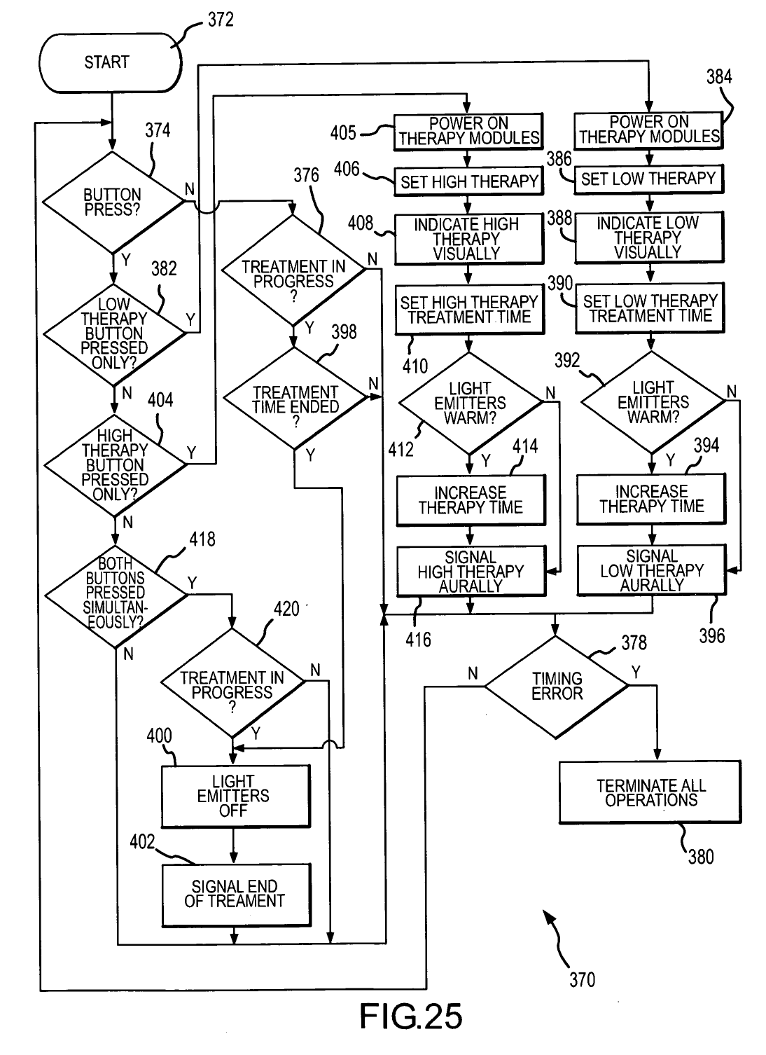

The control module 64 has the general shape of an elongated body 156 formed by joining an upper body shell 158 and a lower body shell 160. The upper body shell 158 and the lower body shell 160 enclose a module circuit board 162 within the interior of the body 156. The module circuit board 162 has electronic circuit components 164 attached to it, including a microprocessor 166, or other microcontroller or electronic controller, which executes a process flow (FIG. 25) for controlling functionality of the therapy device 50. The functionality is established by the user depressing one or both of the low and high power selector buttons 70 and 72.

The low and high selector buttons 70 and 72 are each retained within a guide 168. The guide 168 is attached to the module circuit board 162 and extends from the circuit board 168 upward to the upper body shell 158. Holes 170 are formed in the upper body shell 158 in alignment with the guide 168. The buttons 70 and 72 protrude through the holes 170 above the upper surface of the upper body shell 158. The guide 168 and the holes 170 allow the buttons 70 and 72 to move upward and downward relative to the surface of the upper body shell 158. When the selector buttons 72 and 70 are depressed and move downward, they contact and activate high-power and low-power control switches 174 and 172, respectively, which are also attached to the module circuit board 162 beneath the selector buttons 72 and 70 in the guide 168.

The module circuit board 162 also includes an indicator 176, such as a red light emitting LED and a green light emitting LED, as one of the components 164 of the therapy module 64. The indicator 176 emits one color of light, e.g. amber, to indicate when the therapy device 50 is operating in a low-power mode, another color light, e.g. red, to indicate operation in the high-power mode, and a third color of light, e.g. green, to indicate that a therapy treatment has terminated and that the therapy device 50 is ready to perform the next subsequent treatment. The indicator 176 also blinks to indicate that an error in operation has occurred. An optical guide 178, such as a light pipe, extends from the indicator 176 through a hole 180 in the upper shell 158, to conduct the light from the indicator 176 to the exterior of the body 156. The module circuit board 162 is secured to the lower shell 160 with two fasteners 182.

Electrical power is supplied to the control module 64 by a power cable 184, shown in FIG. 1. A conventional switching power supply 186 supplies DC power from a conventional household-type electrical receptacle to the control module 64. Alternatively, DC electrical power may be obtained from a conventional DC power adapter 188 after it is plugged into a power or cigarette lighter receptacle of a vehicle or connected to a battery. A separate external rechargeable battery power supply (not shown) may also be used to power the control module 64 in place of the adapter 188. A conventional strain relief device 190 connects the power cable 184 to one end of the elongated body 156 of the control module 64.

A therapy module connection receptacle 192 is located on the opposite end of the body 156 from the strain relief 190, as shown in FIGS. 1, 16 and 17. Electrical power and control signals are delivered through the cable 66 to the therapy modules 52 through the connection receptacle 192. A plug 194 (FIG. 1) is connected to the end of the cable 66, and the plug 194 fits into the connection receptacle 192. The receptacle 192 is electrically connected to the module circuit board 162 and is physically attached to the elongated body 156 by fitting within a circular hole 196 created by a semicircular opening defined in an end of the lower body shell 160 and an aligned semicircular opening defined in the upper body shell 158. The circular hole 196 captures and holds the receptacle 192 when the shells 158 and 160 are assembled into the elongated body 156. Both the connection receptacle 192 and the plug 194 have corresponding mating electrical contacts that conduct the control signals, the supply voltage and the reference potential from the module circuit board 162 through the electrical conductors 132, 134 and 136 of the cable 66 (FIG. 10). Alignment structures interact between the receptacle 192 and the plug 194 to ensure that the plug 194 is aligned properly within the receptacle 192 so that the therapy control signal, the supply voltage and the reference potential are applied on the correct conductors 132, 134 and 136, respectively.

An optional attachment clip 198 is connected to the lower body shell 160 for attaching the control module 64 to a belt or other clothing of the user. The attachment clip 198 has wings 200 that attach on opposite sides of the lower body shell 160. Tabs 202 on the wings 200 fit into indentions 204 formed into the lower body shell 160 at the location where the upper and lower body shells 158 and 160 meet when the elongated body 156 is assembled. The attachment clip 198 also has an arm 206 that extends generally parallel to the elongated dimension of the body 156 along the lower surface of the lower body shell 160 to create a space between the lower body shell 160 and the arm 206. A protrusion 208 on the outer end of the arm 206 engages the lower body shell 160. The arm 206 resiliently bends away from the lower body shell 160 to move the protrusion 208 away from the lower body shell 160 so that the user may slide clothing or a belt in between the arm 206 and the lower body shell 160. The resilient characteristic of the arm 206 biases the protrusion 208 toward the lower body shell 160 to retain the clothing or belt against the body shell 160, thereby securing the control module 64 to the clothing or belt.

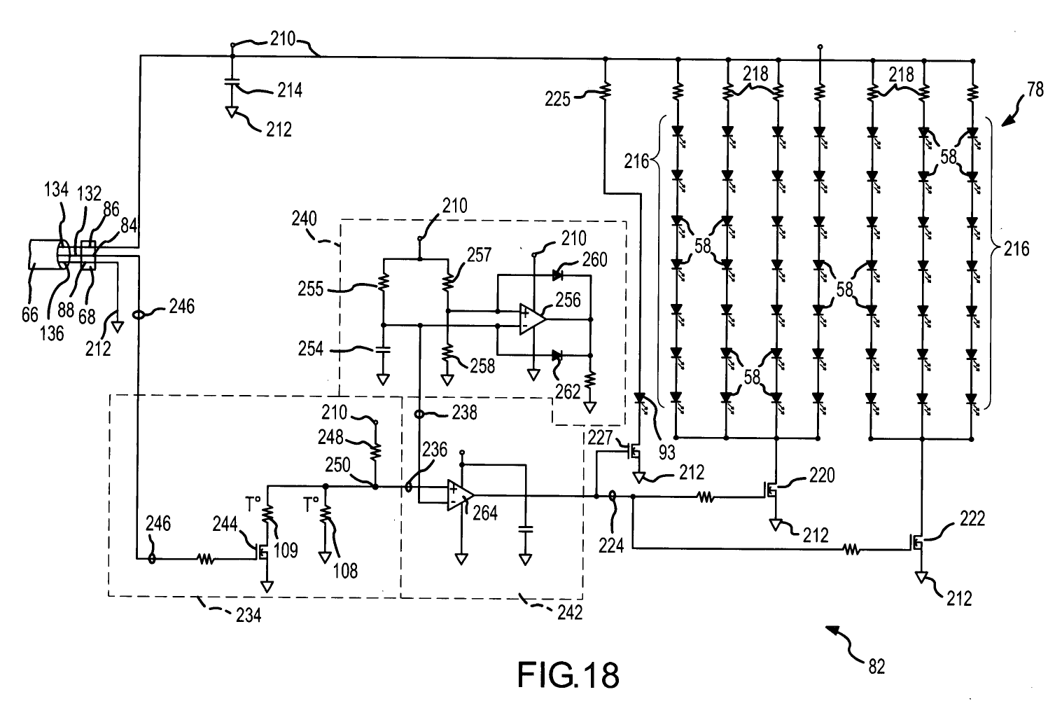

The nature and function of the electrical components 82 which individually control the light delivered from each therapy module 52 are described in conjunction in FIG. 18. The supply voltage for the components 82 of the circuit board 80 is present on conductor 210. The supply voltage on conductor 210 is delivered from the control module 64 on the trace 86 of the flexible circuit 68, and the trace 86 is connected to the electrical conductor 134 of the cable 66 (FIG. 10). Reference potential for the components 82 of the circuit board 80 is present on a reference conductor 212. The reference conductor 212 is connected to the trace 88 of the flexible circuit 68, and the trace 88 is connected to the electrical conductor 136 of the cable 66. A filter capacitor 214 is connected between the conductors 210 and 212 to filter and smooth the supply voltage, thereby ensuring a steady operating voltage between the conductors 210 and 212 for the components 82 of each therapy module 52.

The LEDs 58 of the array 78 are connected in a plurality of columns 216, with a plurality of the LEDs 58 connected in series with one another in each column 216. Seven columns 216 are shown, and seven LEDs are connected in each column 216. The number of columns and the number of LEDs in each column may vary according to the size of each therapy module 52 and the size of its window 60 (FIG. 2).

The LEDs 58 emit light when current is conducted through the series-connected LEDs of each column 216. Current control switches 220 and 222 switch current through the columns 216 of LEDs 58. The switch 220 is connected in series with the four left-hand columns 216, and the switch 222 is connected in series with the three right-hand columns 216 (as shown in FIG. 18). When an LED energization control signal 224 is asserted, the switches 220 and 222 become conductive and conduct current through the columns 216 of LEDs 58, causing them to emit light. When the LED energization control signal 224 is not asserted, the switches 220 and 222 are nonconductive, and no current flows through the columns 216 and no light is emitted from the LEDs 58. Both switches 220 and 222 respond simultaneously to the LED energization control signal 224, causing all the LEDs 58 of the entire array 78 of LEDs on each circuit board 80 within each therapy module 52 to emit light and to cease emitting light simultaneously.

Although two control switches 220 and 222 are shown, a single current control switch could be used in place of the two current control switches 220 and 222, if that single current control switch has sufficient current-carrying capacity to conduct current through all the columns 216 of LEDs 58 simultaneously. Similarly, more than two control switches 220 and 222 could be used to control the current flow through a fewer number of columns 216 of LEDs 58. A resistor 218 is connected in series with the LEDs 58 in each column 216 to limit the current through the LEDs 58 in each column. Connecting the LEDs 58 in series in the columns 216 allows a higher supply voltage to be applied on the conductor 210 than each of the LEDs 58 is individually capable of withstanding.

The energy delivery LED 93 (FIGS. 3 and 4) also responds to the LED energization control signal 224, causing the LED 93 to emit light when the LEDs 58 are energized. The LED 93 is connected through a resistor 225 to the power supply conductor 210. A semiconductor switch 227 becomes conductive upon the assertion of the control signal 224, and current is conducted through the energy delivery LED 93, causing it to emit light. When the control signal 224 is not asserted, the switch 227 is not conductive, no current is conducted through the LED 93, and no light is emitted from the LED 93.

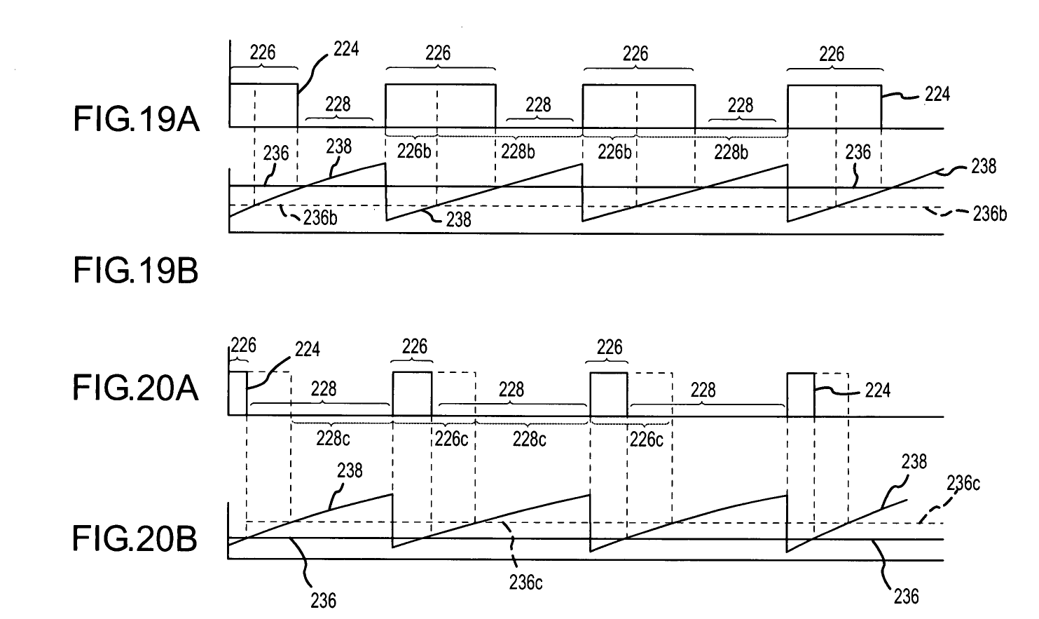

The characteristics of the LED energization control signal 224 are shown in greater detail in FIGS. 19A and 20A. The LED energization control signal 224 is a repeating series of pulses having an on time period 226 separated by an off time period 228. During the on time period 226, the LED energization control signal 224 is asserted and the switches 220 and 222 are conductive. During the off time period 228, the LED energization control signal 224 is deasserted and the control switches 220 and 222 are nonconductive. The amount of heat and light emitted by the LEDs 58 is directly related to the amount of time that the LEDs 58 conduct current during the on time period 226. By regulating the duty cycle of the signal 224, that is the duration of the on time period 226 relative to the sum of the time durations of the on and off time periods 226 and 228, the amount of heat and light emitted by the LEDs 58 is controlled.

The width of the on time period 226 and the off time period 228 of the LED energization control signal 224 is modulated in response to the temperature sensed by the main thermistor 108 during high-power therapy and by both the main and the auxiliary thermistors 108 and 109 during low-power therapy as shown in FIGS. 2-5 and 18. The main and auxiliary thermistors 108 and 109 are part of a temperature reference circuit 234. The temperature reference circuit 234 supplies a temperature reference signal 236 (FIGS. 19B, 20B) that are related to the temperatures sensed by one or both of the thermistors 108 and 109. The temperature reference signal 236 is compared to a triangle signal 238 (FIGS. 19B, 20B) supplied by a triangle waveform generator circuit 240. A comparator circuit 242 compares the temperature reference signal 236 and the triangle signal 238, and creates the on time period 226 and the off time period 228 of the LED energization control signal 224 (FIGS. 19A, 20A) based on that comparison. As the temperature sensed by one or both of the thermistors 108 and 109 changes, the magnitude of the temperature reference signal 236 changes relative to the triangle signal 238, thereby changing the relative amount of time of the on time period 226 and the off time period 228, as shown in FIGS. 19A, 19B and 20A, 20B. The temperature reference circuit 234, the triangle waveform generator circuit 240 and the comparator circuit 242 form a pulse width modulation circuit for modulating the width of the on time and off time periods 226 and 228 of the LED energization control signal 224.

The magnitude of the temperature reference signal 236 is established by one or both of the characteristic resistances of the main thermistor 108 and the auxiliary thermistor 109. The main thermistor 108 and the auxiliary thermistor 109 each exhibit a resistance characteristic that is inversely related to the temperatures that they sense, i.e., their resistances decrease as their temperatures increase. The main thermistor 108 thermally contacts the stud 106 (FIG. 5), preferably through a layer of thermally conductive adhesive (not shown). The direct thermal conductivity through the window 60 from the protrusion 104 to the stud 106 causes the resistance of the main thermistor 108 to be influenced by the temperature of the user's skin. The temperature of the main thermistor 108 is also influenced by the temperature within the housing 56 of the therapy module 52. The auxiliary thermistor 109 responds to the temperature inside of the housing 56. The temperature within the therapy module 52 is created by the heat resulting from the current consumption of the LEDs 58 and the other components 82 (FIG. 5). Some of this heat is transferred through the window 60 as heat therapy to the skin and tissue.

The auxiliary thermistor 109 is electrically connected in parallel with the main thermistor 108 when a semiconductor connection switch 244 is conductive. The connection switch 244 is conductive when a logical high level therapy control signal 246 is asserted as a result of a user depressing the low-power button selector 70 of the control module 64. The control module 64 responds by conducting the therapy control signal 246 on the electrical conductor 132 of the cable 66 and the trace 84 of the flexible circuit 68, from which the therapy control signal 246 is applied to the connection switch 244 of each therapy module 52. The connection switch 244 is nonconductive when a logical low level therapy control signal 246 is asserted. The therapy control signal 246 is at the logical low level when the user depresses the high-power selector button 72 which activates the high-power control switch 174 of the control module 64 (FIGS. 1 and 17).

Thus, whenever the user selects low-power therapy, the therapy control signal 246 is asserted at the logical high level, which causes the connection switch 244 to become conductive and to connect the main and auxiliary thermistors 108 and 109 in parallel with one another in the temperature reference circuit 240. Whenever high-power therapy is selected, the therapy control signal 246 is asserted at the logical low level which causes the connection switch 244 to become nonconductive and to disconnect the auxiliary thermistor 109 from the parallel connection with the main thermistor 108, thereby causing only the main thermistor 108 to have an effect in the temperature reference circuit 240. Consequently, the main and auxiliary thermistors 108 and 109 are connected in parallel to have an effect in the temperature reference circuit 234 only when low-power therapy is selected, and only the main thermistor 108 has an effect in the temperature reference circuit 234 when high-power therapy is selected.

A voltage divider is formed in the temperature reference circuit 234 by a resistor 248 and the main thermistor 108 when the connection switch 244 is not conductive. Under these circumstances, the voltage present at a junction node 250 of the thermistor 108 and the resistor 248 represents a fraction of the supply voltage at 210. That fraction is equal to the resistance of the thermistor 108 divided by the combined resistances of the thermistor 108 and the resistor 248. Connecting the auxiliary thermistor 109 in parallel with the main thermistor 108 when the connection switch 244 is conductive, creates a combined resistance from the parallel-connected thermistors 108 and 109 which is less than the individual resistance exhibited by thermistor 108. Under these circumstances, the voltage at the node 250 is diminished even further, to a fraction of the supply voltage at 210 which is equal to the effective parallel resistance of thermistors 108 and 109 divided by the sum of the resistance of the resistor 248 and the effective parallel resistance of the thermistors 108 and 109. Thus, when high-power therapy is selected, the temperature reference signal 236 will exhibit a greater value than when low-power therapy is selected, as is illustrated by the higher and lower magnitudes of the signal 236 shown in FIGS. 19B and 20B, respectively.

The triangle signal 238 is created by charging and discharging a timing capacitor 254 of the triangle waveform generator circuit 240, shown in FIG. 18. The timing capacitor 254 is charged through a timing resistor 255 that is connected to the voltage supply conductor 210. An inverting input terminal of a comparator 256 is connected to the junction between the timing capacitor 254 and the timing resistor 255. A non-inverting input terminal of the comparator 256 is connected to a junction between reference resistors 257 and 258. The reference resistors 257 and 258 are connected in series between the voltage supply on conductor 210 and the reference potential on conductor 212. As such, the reference resistors 257 and 258 form a voltage divider network that supplies a reference voltage to the non-inverting input terminal of the comparator 256 while the timing capacitor 254 charges and discharges. Diodes 260 and 262 are connected with their polarity supporting conduction from the non-inverting and inverting input terminals to the output terminal of the comparator 256, respectively.

The increasing voltage portion of the triangle signal 238 (FIGS. 19B, 20B) is caused by the increasing voltage across the timing capacitor 254 when that capacitor charges through the timing resistor 255. The capacitor 254 charges when the voltage on the non-inverting input terminal of the comparator 256 is larger than the voltage on the capacitor 254. In this instance, both diodes 260 and 262 are reversed biased as a result of the voltage on the output terminal of the comparator 256 being approximately at the level of the supply voltage at 210. With both diodes 260 and 262 reversed biased, charging current flows through the timing resistor 255 and charges the capacitor 254 and current also flows through the reference resistors 257 and 258.

When the capacitor 254 has charged to a point where its voltage is greater than the voltage supplied by the reference resistors 257 and 258 to the noninverting input terminal of the comparator 256, the output signal of the comparator 256 changes states to approximately the level of the reference potential at 212. At this instant, both diodes 260 and 262 are forward biased and both diodes commence conducting current. The voltage stored across the capacitor 254 is rapidly discharged, as shown by the rapidly decreasing portion of the triangle waveform 238 (FIGS. 19B, 20B). The voltage at the noninverting input terminal to the comparator 256 is immediately reduced to the forward biased voltage across the diode 262, which is typically about 0.7 volts greater than reference potential at conductor 212. The time required to discharge the capacitor 254 to the voltage of the voltage drop across the diode 262 is somewhat greater than the almost instantaneous change of the voltage at the noninverting input to the comparator 256. In this relatively short but nevertheless finite amount of time, the voltage across the capacitor 254 decreases, thereby decreasing the triangle signal 238. The timing capacitor 254 discharges until its voltage reaches the voltage at the noninverting input to the comparator 256, at which point the voltage at the output terminal of the comparator 256 again assumes the level of the supply voltage at 210, causing the voltage of the triangle signal 238 to commence increasing as a result of the capacitor 254 charging through the resistor 255.

The process of charging and discharging the timing capacitor 254 continues in the manner described, thereby creating the triangle signal 238 from the voltage across the timing capacitor 254. The rates at which the capacitor 254 is charged and discharged remain essentially the same from one cycle of the triangle signal 238 to the next cycle. Consequently, each cycle of the triangle signal 238 has essentially the same wave shape. Furthermore, the frequency of the triangle signal 238 is also constant due to the consistent shape of each cycle. In the preferred embodiment, the triangle signal 238 has a frequency of about 1 kHz.

The triangle signal 238 and the temperature reference signal 236 are compared to one another in the comparator circuit 242 in order to derive the LED energization signal 224. The comparison is performed by a comparator 264. The temperature reference signal 236 is applied to a noninverting input terminal of the comparator 264 and the triangle signal 238 is applied to the inverting input terminal of the comparator 264. Whenever the voltage of the temperature reference signal 236 is greater than the voltage of the triangle signal 238, the output terminal of the comparator 264 assumes a logic high level to create the on time period 226 (FIGS. 19A, 20A) of each pulse of the LED energization signal 224. The switches 220, 222 and 227 respond to the on time period 226 by conducting current through the LEDs 58 and 93, thereby causing the array 78 of LEDs to emit heat and light and causing the power delivery LED 93 to signal that energy is being delivered. Whenever the voltage of the temperature reference signal 236 is less than the voltage of the triangle signal 238, the voltage at the output terminal of the comparator 264 assumes a logic low level to create the off time 228 (FIGS. 19A, 20A) of each pulse of the LED energization signal 224. The current switches 220, 222 and 227 respond to the off time period by ceasing to conduct current through the LEDs 58 and 93, thereby terminating the heat and light emission from the array 78 and indicating that the therapy module is no longer delivering therapeutic energy.

The change in the amount of light emitted between high-power therapy and low-power therapy is understood by comparing FIGS. 19A, 19B with FIGS. 20A, 20B. The temperature reference signal 236 is of increased magnitude when high-power therapy is selected, because the auxiliary thermistor 109 is not connected in parallel with the main thermistor 108 in the temperature reference circuit 234, as shown in FIG. 19B. Similarly, the temperature reference signal 236 is of decreased magnitude when low-power therapy is selected, as shown in FIG. 20B, because the auxiliary thermistor 109 and the main thermistor 108 are connected in parallel. The higher level of the temperature reference signal 236 intersects with the triangle signal 238 to create relatively longer on time periods 226 (FIG. 19A) compared to the relatively shorter on time periods 226 (FIG. 20A) resulting when the lower-level temperature reference signal 236 intersects with the triangle signal 238. The longer on time periods 226, resulting from selecting high-power therapy, deliver more heat and light energy to the tissue of the user in a specific amount of time, while the relatively shorter on time periods 226, resulting from selecting low-power therapy, deliver less heat and light energy to the tissue of the user in the same specific amount of time. Moreover, the width of the on time period 226 and the off time period 228 are regulated in response to temperature in both the high-power therapy and the low-power therapy. As a consequence, the temperature of the tissue is regulated. Temperature regulation occurs in the same manner when either high-power therapy or low-power therapy is selected; only the regulated temperature varies.

The effect of an increase in temperature beyond the regulated temperature is illustrated in FIGS. 19A and 19B. The main thermistor 108 senses the increased temperature and its resistance decreases because of its inverse relationship characteristic of temperature and resistance. The decreased resistance of the thermistor 108 decreases the magnitude of the temperature reference signal 236 to the level shown by the dashed lines 236b in FIG. 19B. The decreased value of the temperature reference signal 236b interacts with the triangle signal 238 to decrease the on time of the pulses of the LED energization signal 224 to the amount shown by the dashed lines 226b and increases the off time to amount shown by the dashed lines 228b. Consequently, the decreased on time periods of the pulses of the LED energization signal 224 reduce the amount of heat and light energy delivered by the LEDs 58 to the skin, and the reduced amount of light diminishes the temperature of the skin. Simultaneously, the intensity of the light emitted from the energy delivery LED 93 diminishes, because the LED 93 is not conductive as much as it was previously. The diminished intensity of light from the LED 93 visually indicates to the user that a reduction in energy delivery has occurred.

Conversely, a decreased temperature causes the temperature reference signal 236 to be higher, as shown in FIGS. 20A and 20B. Although the circumstance shown in FIGS. 20A and 20B results from low-power therapy, the same effect also occurs during high-power therapy, as a result of the response of only the main thermistor 108. As shown in FIGS. 20A and 20B, the main and auxiliary thermistors 108 and 109 respond to the decreased temperature but the response is moderated by the effect of the parallel connected thermistors 108 and 109. A decrease in temperature sensed by the thermistors 108 and 109 causes the temperature reference signal 236 to increase, due to the inverse characteristic relationship of resistance and temperature of the thermistors 108 and 109. The increased resistance of the thermistors 108 and 109 increases the magnitude of the temperature reference signal 236 to the level shown by the dashed lines 236c in FIG. 20B. The increased value of the temperature reference signal 236c interacts with the triangle signal 238 to increase the on time period of the pulses of the LED energization signal 224 to the amount shown by the dashed lines 226c and decreases the off time period to the amount shown by the dashed lines 228c. Consequently, the on time periods of the pulses of the LED energization signal 224 increase, increasing the amount of heat and light delivered by the LEDs 58 to the skin. The increased amount of heat and light increases the temperature of the skin. The intensity of the light emitted from the energy delivery LED 93 increases, signaling to the user an increase in the power delivered.

In this way, the heat and light emitted from the LEDs 58 is regulated in relation to the temperature of the skin. An increase in skin temperature is related to an increase in temperature within the therapy module 52, and the temperature increase results in a decrease in the amount of heat and light delivered to the skin during a given time period. Conversely, a decrease in skin temperature is related to a decrease in temperature within the therapy module 52 and results in an increase in the amount of heat and light delivered during a given time period, thereby elevating the skin temperature until a desired temperature is reached. This same temperature regulating effect occurs with both high and low therapy treatment. However when low-power therapy is selected, less light energy is delivered in a given time period.

The desired temperature of the skin at which this regulation occurs is established by adjusting the relative resistance values of the resistor 248 and the thermistors 108 and 109, shown in FIG. 18. Adjusting these values causes the temperature reference signal 236 to be increased or decreased slightly in value. The value of the temperature reference signal 236 sets the temperature at which the regulation occurs. The resistance values of the resistor 238 and the thermistors 108 and 109 are adjusted empirically to account for the thermal mass of each therapy module 52, the transmissivity of the LEDs 58, the heating effect of the LEDs 58, and other factors which are specific to the therapy modules 52. However, once established, the resistance values of the resistor 238 and the thermistors 108 and 109 can be used for all substantially identical therapy modules 52.

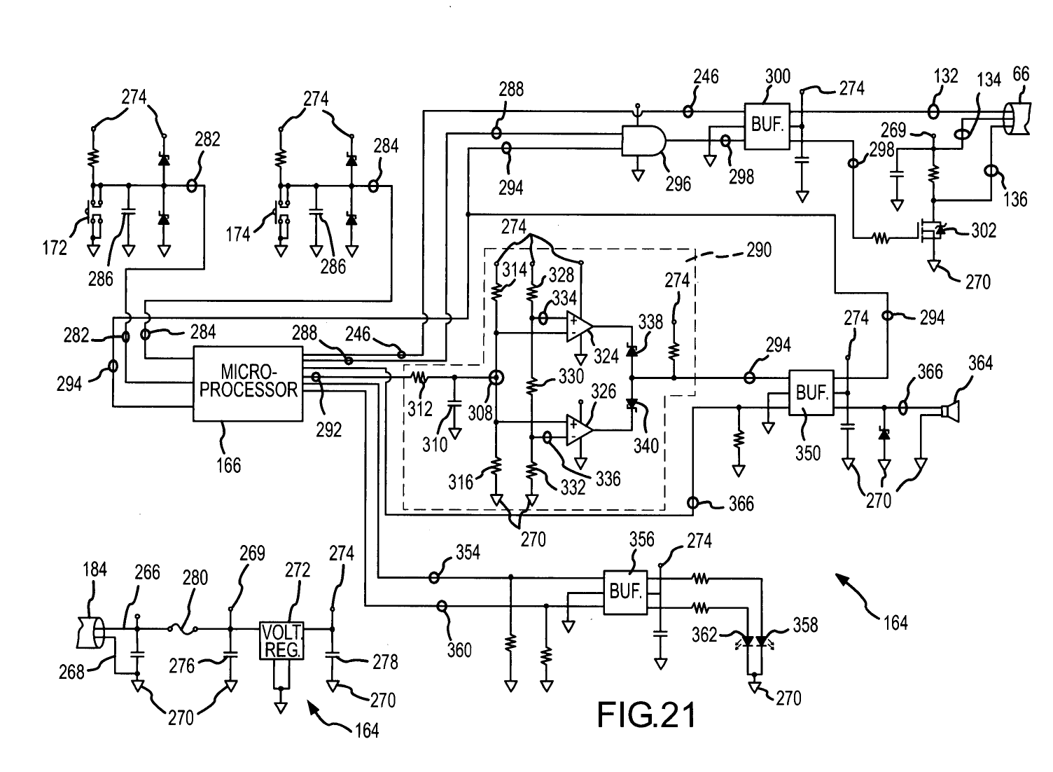

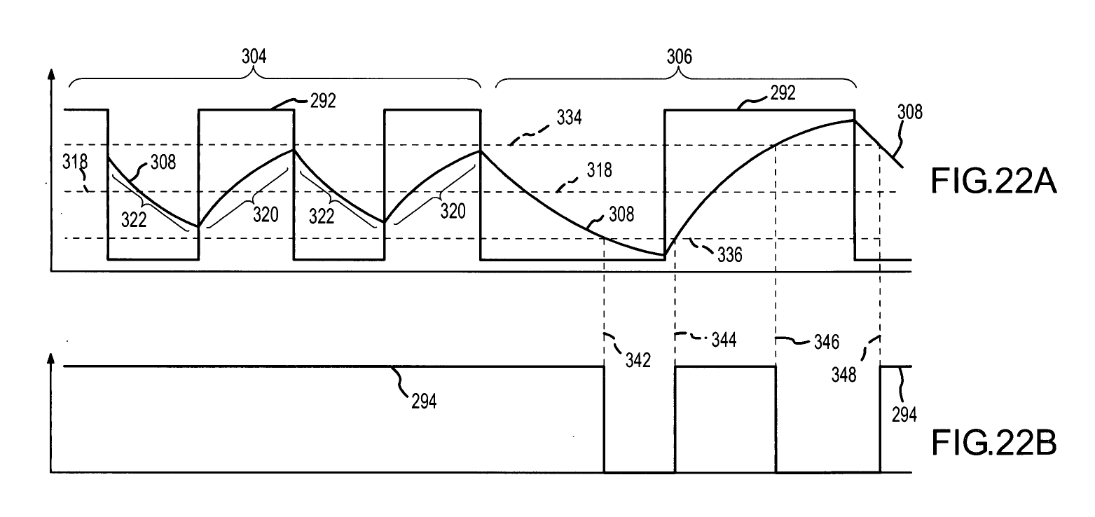

Regardless of whether high-power therapy or low-power therapy is selected by the user, the therapy device 50 preferably delivers a relatively constant amount of light energy to the user during each treatment. The desired amount of light energy to be delivered during each treatment is approximately 5-8 Joules/square centimeter of skin surface area. To deliver this amount of light energy when low-power therapy is selected and the on time period 226 of the LED energization control signal 224 is relatively shorter, the time duration of the entire treatment is increased. When high-power therapy is selected and the on time period 226 of the control signal is relatively longer, the time duration of the entire treatment is decreased. In many cases, the desired amount of light energy will be delivered before the maximum regulated temperature of the skin will be reached. Controlling the time duration of the treatment is one of the primary functions of the components 164 attached to the circuit board 162 of the control module 64, shown in FIGS. 21 and 22.

The electronic components 164 of the control module 64 include the microprocessor 166, or other controller, which establishes and controls the overall functionality of the control module 64, as shown in FIG. 21. In general, those functions include controlling the application of power to the therapy modules 52 in response to the user selecting either high-power or low-power therapy treatments, measuring or timing the duration of the treatment depending upon whether the high-power or low-power therapy has been selected, adjusting the timing or compensating for the timing of the high-power or low-power therapy treatment to account for the temperature of the LEDs 58 when the treatment starts, monitoring an internal timing or clock function to prevent errors which could lead to treatments of an unintended time duration, and signaling various operational conditions and status of the therapy device 50.

As shown in FIG. 21, electrical power for the components 164 of the control module 64 and for the electronic components 82 of each therapy module 52 (FIG. 18) is supplied through the power cable 184 by the conventional switching power supply 186 or the DC power adapter 188 (FIG. 1). A relatively high DC voltage, for example 12 volts, is supplied on one conductor 266 of the cable 184, and the reference potential is supplied on another conductor 268 of the cable 184. The DC voltage is conducted within the control module 64 on a high voltage supply conductor 269. The reference potential is applied on a reference potential conductor 270 within the control module 64. Electrical power is delivered to the therapy modules 52 by directly connecting the high voltage supply conductor 269 to the conductor 134 of the cable 66 and by selectively connecting the reference potential conductor 270 through a switch 302 to the conductor 136 of the cable 66. The conductors 134 and 136 are connected to the traces 86 and 88 of the flexible circuit 68, which are connected to the supply and reference conductors 210 and 212 of the therapy modules 52 (FIGS. 1, 10 and 18). Current from the high voltage supply on conductor 269 is conducted through the therapy modules 52 when the switch 302 is conductive, thereby completing a circuit from the high voltage supply on conductor 269 through the therapy modules 52 to the reference potential on conductor 270.

A voltage regulator 272 receives the DC voltage from the conductor 269. The voltage regulator 272 creates a relatively low DC voltage, for example 5 volts, which is supplied on a control module voltage supply conductor 274 to power the electronic components 164 of the control module 64. A filter capacitor 276 connects between the relatively higher DC voltage on the conductor 269 (also on conductor 134 and trace 86) and control module reference potential conductor 270 (also conductor 136 and trace 88). Another filter capacitor 278 connects between the relatively lower DC voltage on the control module voltage supply conductor 274 and the reference potential conductor 270. The filter capacitors 276 and 278 smooth the magnitude of the applied voltages. Over current protection is provided by a fuse 280.

The application of electrical power to the therapy modules 52 occurs in response to the user selecting either low-power therapy treatment or high-power therapy treatment by closing the low-power control switch 174 (FIG. 17) or the high-power control switch 172, also shown in FIG. 22. The simultaneous closure of both control switches 172 and 174 causes the control module 64 to cease delivering electrical power to the therapy modules 52, thereby terminating the therapy treatment. A low-power control signal 284 is asserted to the microprocessor 166 when the low-power control switch 174 is closed. A high-power control signal 282 is asserted to the microprocessor 166 when the high-power control switch 172 is closed. Debounce capacitors 286 filter out any transient or spurious portions of the control signals 282 and 284 caused by the closure of the control switches 172 and 174. When the low-power and the high-power control signal 284 and 282 are not asserted, the signals 284 and 282 assume the level of the low DC supply voltage on conductor 274.

The microprocessor 166 responds to the low-power and high-power control signals 284 and 282 to apply electrical power to the therapy modules, to measure the time duration of the selected high-power or low-power therapy treatment, and to commence monitoring an internal timing or clock function to prevent timing errors, among other things. The assertion of either control signal 282 or 284 causes the microprocessor 166 to supply an enable signal 288. The enable signal 288 enables the delivery of electrical power to the therapy modules for the time duration of the selected therapy treatment. The deassertion of the enable signal 288 terminates the delivery of electrical power to the therapy modules 52 and thereby terminates the treatment. The simultaneous assertion of the low-power and high-power control signals 284 and 282 causes the microprocessor 166 to deassert the enable signal 288, because the closure of control switches 172 and 174 indicates that the user has elected to terminate the treatment.

A clock monitoring circuit 290 responds to a clock signal 292 from the microprocessor 166 and supplies a watchdog signal 294 so long as the clock signal 292 represents substantially regular and accurate timing. Should an internal timing malfunction within the microprocessor 166 occur, the amount of time for the selected therapy treatment would be altered, because the microprocessor 166 establishes the length of the selected therapy treatment based on the frequency of the clock signal 292. The clock signal 292 should have a normal, regular and predetermined frequency. The clock monitoring circuit 290 asserts a watchdog signal 294 while the clock signal 292 exhibits its regular and predetermined timing, and the clock monitoring circuit 290 deasserts the watchdog signal 294 should any significant decrease in the frequency of the clock signal 292 occur. The assertion of the watchdog signal 290 signifies correct, accurate or acceptable internal timing.

Electrical power is delivered to the therapy modules 52 only when both the enable signal 288 and the watchdog signal 294 are simultaneously asserted. The enable signal 288 and the watchdog signal 294 are applied to an AND gate 296. The simultaneous assertion of the signals 288 and 294 to the AND gate 296 causes it to deliver a power delivery control signal 298 to a buffer 300. The buffer 300 conducts the power delivery control signal 298 to a power control switch 302, which becomes conductive in response to the assertion of the power delivery control signal 298. When conductive, the power control switch 302 electrically connects the conductor 136 in the cable 66 to the control module reference potential conductor 270. The conductor 134 in the cable 66 is connected to the high voltage supply conductor 269. With the switch 302 conductive, electrical power is conducted to the therapy modules 52 from the high voltage power supply conductor 269, through the conductor 134 of the cable 66, through the trace 86 of the flexible circuit 68 to the supply voltage conductor 210 of each therapy module, through the components 82 of the therapy module 52, from the reference voltage conductor 212, through the trace 88 of the flexible circuit 68, through the conductor 136 of the cable 66, and through the conductive power delivery switch 302 to the control module reference potential conductor 270.

The microprocessor 166 supplies the therapy control signal 246 in response to the assertion of the low-power control signal 284 or the high-power control signal 282. The therapy control signal 246 is a logical high level signal when the control signal 284 indicates that the user has selected low-power therapy by closing the control switch 174. The therapy control signal 246 is a logical low level signal when the control signal 282 indicates that the user has selected high-power therapy by closing the control switch 172. The therapy control signal 246 is applied to the buffer 300 and is conducted through the buffer 300 onto the conductor 132 of the cable 66. The therapy control signal 246 is conducted on the conductor 132 to the trace 84 of the flexible circuit 68, and from the trace 84 to the thermistor connection switch 244 to each therapy module 52 (FIG. 18). The logical high level therapy control signal 246, resulting from selecting low-power therapy, causes the auxiliary thermistor 109 to be connected in parallel with the main thermistor 108, thereby reducing the duty cycle of heat and light delivery. The logical low level therapy control signal 246, resulting from selecting high-power therapy, causes only the main thermistor 108 to establish the higher duty cycle of heat and light delivery, as has been discussed previously in conjunction with FIG. 18.

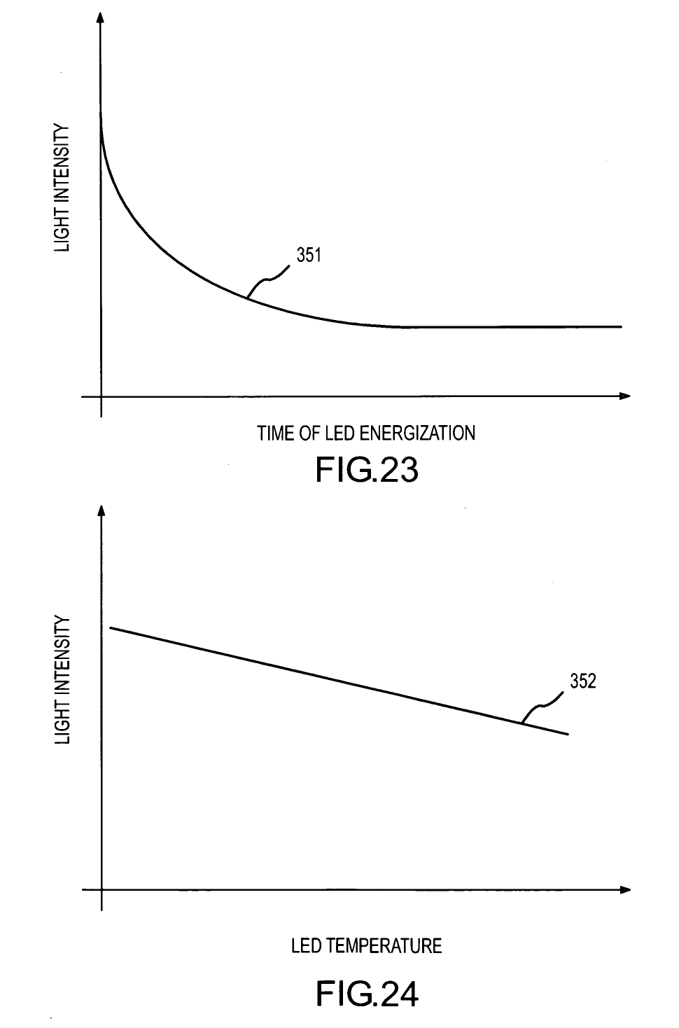

The clock monitoring circuit 290 responds to the frequency of the clock signal 292 to determine whether the internal timing within the microprocessor 166 is occurring as intended. As shown at 304 in FIG. 22A, the normal clock signal 292 is a repeating square wave signal having a predetermined fixed frequency and a duty cycle of approximately 50 percent. As a result of the 50 percent duty cycle, the clock signal at 304 is at a logical high level for one half of each normal cycle and is at a logical low level for the remaining one half of each normal cycle. Should the clock signal 292 deviate from these normal conditions, as illustrated at 306 in FIG. 23A, the frequency changes compared to the normal frequency, and the amount of time that the clock signal 292 occupies the logical low level or the logical high level will deviate from the normal conditions illustrated at 304. The clock monitoring circuit 290 responds to the abnormal condition illustrated at 306 by deasserting the watchdog signal 294.

The normal or abnormal conditions of the clock signal 292 are reflected by a voltage or frequency-related signal 308 developed across a filter capacitor 310, as shown in FIG. 21. The clock signal 292 is conducted through a filter resistor 312 from the microprocessor 166 to the filter capacitor 310. Also connected to the filter capacitor 310 are a pair of resistors 314 and 316 which are connected in series between the control module supply conductor 274 and the control module reference potential conductor 270. The resistors 314 and 316 form a voltage divider, the midpoint of which is approximately half way between the DC voltage on the supply conductor 274 and the reference potential on conductor 270. By connecting the filter capacitor 310 to the junction of the resistors 314 and 316, the filter capacitor 310 will normally charge to a voltage approximately halfway between the supply voltage on conductor 274 and the reference potential on the conductor 270. The normal voltage of the filter capacitor 310 created by the voltage divider resistors 314 and 316 and is shown at 318 in FIG. 22A.

The application of the normal clock signal 292 to the filter capacitor 310 has the effect of charging the filter capacitor 310 to a voltage higher than its normal state during the on or logical high time periods of the clock signal 292, as shown at 320 in FIG. 22A. During its on or logical high time periods, the potential of the clock signal 292 is approximately at the level of the DC voltage on the supply conductor 274 (FIG. 21). During the off or logical low time periods of the clock signal 292, the filter capacitor 310 is discharged to a voltage lower than its normal state, as shown at 322 in FIG. 22A. The potential of the clock signal 292 during the off or logical low time periods is approximately at the reference potential on conductor 270. Thus, the frequency-related signal 308 across the filter capacitor 310 increases above the normal voltage 318 during the on time periods of the clock signal 292 and decreases below the normal voltage 318 during the off time periods of the clock signal 292, as shown in FIG. 22A. The amounts of increase and decrease are directly related to the normal frequency of the clock signal 292.

Should the frequency of the clock signal 292 decrease as shown at 306 in FIG. 22A, the amount of time during which the filter capacitor 310 can charge and discharge is increased. As a consequence, the signal 308 across the filter capacitor 310 increases and decreases to a greater amount relative to the normal voltage 318 during the abnormal conditions 306, compared to the normal conditions at 304. A reduction in the frequency of the clock signal 292 would signify that the microprocessor 166 is operating more slowly, because more time passes with each complete cycle of the abnormal clock signal 292. Operating more slowly would have the effect of increasing the time duration of the therapy treatment, and increased therapy treatments may result in delivering heat and light energy to the tissue for longer-than-intended time. Consequently, the main concern is to detect malfunctions within the microprocessor 166 which result in a decreased frequency of the clock signal 292, which is the case illustrated at 306 in FIG. 22A.

Unacceptable excursions, both high and low, of the frequency-related signal 308 are detected by comparators 324 and 326 that are part of a window comparator circuit. A voltage divider formed by resistors 328, 330 and 332 is connected between the control module supply conductor 274 and the control module reference potential conductor 270. The resistors 328, 330 and 332 divide the voltage between the supply conductors 274 and 270 into an upper-level comparison voltage 334 and a lower-level comparison voltage 336 (FIG. 22A). The values of the resistors 328, 330 and 332 are selected to establish the level of the comparison voltages 334 and 336 at levels which are slightly greater than the maximum magnitude of the frequency-related signal 308 and slightly less than the minimum magnitude of the frequency-related signal 308, respectively, as shown in FIG. 22A. Thus, the normal voltage excursions of the frequency-related signal 308 during normal conditions 304 of the clock signal 292 cause the voltage of the frequency-related signal 308 to remain below the upper comparison voltage 334 and above the lower comparison voltage 336. However, during abnormal conditions 306, the frequency-related signal 308 may increase above the upper comparison voltage 334 and/or decrease below the lower comparison voltage 336.

The comparators 324 and 326 detect when the frequency-related signal 308 exceeds the upper comparison voltage 334 and falls below the lower comparison voltage 336. The frequency-related signal 308 is supplied to the inverting input terminal of the comparator 324 and to the noninverting input terminal of the comparator 326. The upper comparison voltage 334 is supplied to the noninverting input terminal of the comparator 324, and the lower comparison voltage 336 is supplied to the inverting input terminal of the comparator 326. So long as the frequency-related signal 308 remains less than the upper comparison voltage 334, the comparator 324 supplies a logic high signal on its output terminal. So long as the frequency-related signal 308 remains greater than the lower comparison voltage 336, the comparator 326 also supplies a logic high signal on its output terminal. The two logic high output signals reverse bias the two Schottky diodes 338 and 340, which are connected to the output terminals of the comparators 324 and 326, respectively. Consequently, the watchdog signal 294 assumes a logic high level whenever the frequency-related signal 308 remains within its normal excursion levels between the upper and lower comparison voltages 334 and 336. A logic high level of the watchdog signal 294 therefore indicates normal functionality of the clock signal 292.

Under abnormal conditions 306 (FIG. 22A) the frequency-related signal decreases below the lower comparison voltage 336, causing the comparator 326 to supply a logic low signal at its output terminal. The logic low signal at the output terminal of the comparator 326 forward biases the Schottky diode 340, and causes the watchdog signal 294 to assume a logic low level as shown in FIG. 22B. As shown in FIG. 22A, the frequency-related signal 308 decreases below the lower comparison voltage 336 at the time 342, immediately causing the watchdog signal 294 to assume the logic low level. The watchdog signal 294 remains at the logic low level until the frequency-related signal 308 begins to increase as a result of charging during the on time period of the clock signal 292. Once the frequency-related signal 308 increases to the lower comparison voltage 336, as shown at time 344, the watchdog signal 294 assumes its normal logic high level. As the frequency-related signal 308 continues to increase during the abnormal conditions 306, it exceeds the upper comparison voltage 334 at time 346, causing the comparator 324 to supply a logic low signal at its output terminal. The Schottky diode 338 is forward biased, causing the watchdog signal 294 to again assume the logic low level, as shown in FIG. 22B. During the off time period of the abnormal clock signal 292 shown at 306, the frequency-related signal 308 decreases until it reaches the upper comparison voltage 334 at time 348, causing the comparator 324 to again supply the logic high signal watchdog signal 294 at its output terminal.

In the manner described, any significant deviation of the clock signal 292 from its normal frequency will result in the comparators 324 and 336 causing the watchdog signal 294 to assume a logic low level, thereby deasserting the watchdog signal 294. The watchdog signal 294 is supplied to a buffer 350, which conducts the watchdog signal to the AND gate 296 and back to the microprocessor 166. When the watchdog signal 294 is deasserted, the AND gate 296 terminates the delivery of the power delivery control signal 298. Thus the therapy modules 52 are deprived of electrical power during the abnormal portions of the clock signal 292. When the frequency-related signal 308 returns to the values between the upper and lower comparison voltages 334 and 336, the watchdog signal 294 is again asserted (FIG. 22A). In this manner, the electrical power delivery to the therapy modules 52 is limited to no more than that which would occur if the clock signal 292 was operating at its normal predetermined frequency. Nevertheless, the deassertion of the watchdog signal 294 on any regular basis indicates a serious problem which must be recognized and dealt with by the microprocessor 166.

The microprocessor 166 recognizes serious problems with reoccurring deassertions of the watchdog signal 294 by counting the number of times that the watchdog signal is deasserted within a predetermined amount of time. If the microprocessor detects that the watchdog signal 294 has been deasserted more than a predetermined number of times within the predetermined amount of time, the microprocessor 166 permanently deasserts the enable signal 288 to terminate the treatment. Although the microprocessor will not be able to accurately determine the predetermined amount of time during which it counts deassertions of the watchdog signal 294, due to the abnormal conditions of the clock signal 292, the frequency of the clock signal 294 is so large in comparison to the deviation in the counted predetermined amount of time that an accurate indication of the proper functionality of the internal microprocessor clock can still be obtained. Reasonable accuracy is also enhanced by the fact that two deassertions of the watchdog signal 294 will typically occur during each cycle of the abnormal clock signal 292, as understood from FIG. 23A.

The microprocessor 166 also times the duration of the high-power and the low-power therapy treatments. The enable signal 288 is asserted for the entire duration of each therapy treatment, and is deasserted at the conclusion of each therapy treatment. The basic time duration of each high-power therapy treatment and each low-power therapy treatment is preestablished. A lesser amount of light energy is delivered during the low-power therapy treatment due to the shorter on time 226 of the LED energization control signal 224, compared to the longer on time 226 of the LED energization control signal 224 during high-power therapy treatment (FIGS. 19A and 20A). Because the amount of light energy delivered during the low-power therapy treatment in a given amount of time is less than the amount of light energy delivered during the high-power therapy treatment in the same given amount of time, the total time duration of the low-power therapy treatment must be longer than the total time duration of the high-power therapy treatment if the same amount of light energy is delivered to the skin and tissue in both treatments. The microprocessor 166 establishes the length of the high-power and low-power therapy treatments based on the typical on time of the LED energization control signal 224 for each type of therapy treatment. The timing for the enable signal is initiated in response to the assertion of the low-power control signal 282 or the high-power control signal 284.

In addition to establishing the basic time duration of each high-power and each low-power therapy treatment, the microprocessor 166 also increases the length of the basic time duration of each therapy treatment in relation to the time which has expired since the last therapy treatment. The adjustment to the basic time duration of each therapy treatment is to compensate for the estimated temperature of the LEDs 58 at the time that the next subsequent therapy treatment commences. As shown by the curve 351 shown in FIG. 23, the light intensity from an LED diminishes after that LED has been initially energized. The initial reduction in light intensity is relatively rapid beginning immediately after the LED has been first energized, assuming that the LED initially is at room temperature. As the LED conducts energy, it begins to heat. The heat causes diminished light intensity from the LED, and that diminished intensity may be accompanied by a shift in the wavelength of light emitted from the LED. Typically that shift will be toward a shorter wavelength and a corresponding higher frequency. The curve 352 shown in FIG. 24 illustrates that increasing temperature of the LED results in a diminished intensity of light emitted by the LED. At some point, however, the temperature of the LED stabilizes and its light intensity also stabilizes, as shown in FIG. 23.

A diminished intensity of emitted light from the LEDs 58 results in a diminished amount of light energy transferred to the tissue. LEDs which have an elevated temperature at the beginning of each therapy treatment will not deliver as much light energy, as shown by graphs 351 and 352 in FIGS. 23 and 24. Consequently, to deliver a predetermined amount of light energy during each therapy treatment, additional time must be added to the basic time for each of therapy treatment to compensate for LEDs which have an elevated temperature at the commencement of the next subsequent therapy treatment.

The LEDs 58 will have an elevated temperature at the commencement of a therapy treatment if the therapy device 50 has been used relatively recently in an earlier therapy treatment. If a relatively long time has elapsed since the earlier therapy treatment, for example approximately ten minutes, it is presumed that the LEDs 58 have cooled sufficiently from the elevated temperature attained during the earlier therapy treatment so that their temperature approximates room temperature. Under such circumstances, no additional time will be added to the basic time for the next subsequent therapy treatment.

The microprocessor 166 determines whether to add additional time to the normal time duration of the next subsequent therapy treatment if the previous therapy treatment ended within a predetermined amount of time before the next subsequent therapy treatment is initiated. The predetermined amount of time between the previous and the following therapy treatment is approximately ten minutes, which is the amount of time during which it is presumed that the LEDs 58 will cool to room temperature. Therefore, if the next subsequent treatment commences more than ten minutes after the termination of the previous treatment, the microprocessor 166 does not add additional time to the basic time duration of the therapy treatment. Not adding additional time assumes that the LEDs have cooled sufficiently so as to account for the increased intensity of light delivered when the LEDs are initially powered from a relatively cool state, as understood from FIG. 23. On the other hand, if the next subsequent treatment commences less than ten minutes after the termination of the previous treatment, the microprocessor 166 adds additional time to the basic time duration of the therapy treatment to compensate for the reduced intensity of light delivered because the LEDs are initially powered while in a warm state.

When the therapy device 50 is available for use, the microprocessor 166 asserts a first indication signal 354 to a buffer 356. The buffer 356 delivers the first indication signal to a LED 358, causing the LED 358 to emit light. The LED 358 preferably emits a green color of light, which indicates that the device 50 is ready for use. During high-power therapy treatments, a second indication signal 360 is asserted to the buffer 356. The buffer 356 delivers the second indication signal to an LED 362. The LED 362 preferably emits a red color of light, which indicates that high-power treatment therapy has been selected and is progressing. During low-power therapy treatments, both the first and second indication signals 354 and 360 are simultaneously asserted to the buffer 356, and both indication signals 354 and 360 cause the LEDs 358 and 362 to emit light simultaneously. A green light from the LED 358 and a red light from the LED 362 combine to form an amber color, which signifies that low-power treatment therapy has been selected and is progressing. The microprocessor 166 indicates the end of a treatment by asserting the first indication signal 354, indicating that the therapy device 50 is again ready for use. In addition, the energy delivery LED 93 (FIGS. 3, 4 and 18) will cease emitting light at the end of a treatment, and that light will no longer be visible through the translucent upper shell 74 (FIGS. 3 and 4). Signaling the end of a therapy treatment is helpful because the user is not likely to recognize immediately that the therapy has ended, since the therapeutic light is not visible to the user and the heat from the therapy modules will not have dissipated significantly for the user to recognize a physiological decrease in temperature. A timing error or malfunction is indicated by delivering one or both of the indication signals 354 or 360 in a repeating pulse-like pattern, causing one or both of the LEDs 358 and 362 to blink. The LEDs 358 and 362 are part of the indicator light 176 on the control module circuit board 162 (FIG. 17).

The therapy device 50 also includes a speaker 364 by which to aurally indicate the occurrence of certain events. The speaker 364 is energized by a speaker signal 366 supplied by the microprocessor 166 through the buffer 350. The speaker signal 366 generates an audible beep from the speaker 364. A single beep is delivered when the low-power treatment begins, a double beep is delivered when the high-power treatment begins, and three beeps are delivered when either the high-power or the low-power therapy treatment ends.

The functionality of the microprocessor 166 in performing the previously-described tasks and in controlling the general operation of the therapy device 50 is illustrated and discussed in conjunction with a process flow 370, shown in FIG. 25. The process flow 370 is executed as a result of the microprocessor 166 (FIG. 21) performing programmed instructions. Each of the steps or events of the process flow 370 are designated by separate reference numbers.

The process flow 370 begins at 372 where the microprocessor 166 is powered up and performs a self test and initialization. After powering up and initializing at 372, the microprocessor 166 determines at 374 if one of the low-power or high-power control switches 174 or 172 (FIG. 21) has been closed as a result of depressing the low-power selector button 70 or the high-power selector button 72 (FIGS. 1 and 17). If one or both of the buttons 70 or 72 has been pressed, one or both of the control signals 282 or 284 (FIG. 21) is at a logical low level. If it is determined at 374 that neither of the buttons 70 or 72 have been pressed, the program flow 370 advances to 376, where it is determined whether a treatment is already in progress as a result of the enable signal 288 (FIG. 21) being asserted.

If no treatment is in progress when the determination is made at 376, the process flow 370 proceeds to 378, where a determination is made if a clock timing error has occurred. The clock monitoring circuit 290 and the microprocessor 166 (FIG. 21) determine whether a clock timing error has occurred in the manner previously described. If it is determined at 378 that a clock timing error has occurred, all operations of the microprocessor 166 are terminated, as shown at 380. All operations cease as a result of the microprocessor deasserting the enable signal 288 (FIG. 21). Visual and aural indications are also delivered by the LEDs 358 and 362 and the speaker 364 (FIG. 21). If no timing error is determined to have occurred at 378, the process flow 370 enters a sensing loop around 374, 376 and 378 until a power selector button 70 or 72 is pressed as determined at 374 or until a timing error occurs as determined at 378.

When one of the power selector buttons 70 or 72 has been pressed, the process flow 370 passes from 374 to 382 where the determination is made as to whether or not the low-power selector button 72 was the only button that was pressed. If the only button pressed was the low-power selector button 72, the process flow 370 proceeds to 384 where electrical power is supplied to the therapy modules 52 by the closure of the power delivery switch 302 (FIG. 21). After the therapy modules 52 are electrically powered, the process flow 370 moves to 386 where the microprocessor 166 asserts the high-level therapy control signal 246 (FIGS. 18 and 22), to cause the therapy modules 52 to deliver low-power treatment. After the therapy modules 52 are set to the low-power treatment at 386, the process flow 370 proceeds to 388, where the low-power therapy treatment is visually indicated by the LEDs 358 and 362 (FIG. 21) of the control module 64 and by the power delivery LED 93 of each therapy module 52.

The program flow continues from 388 to 390 where the microprocessor 166 starts an internal timer to count down the basic treatment time for the low-power treatment. Thereafter at 392, a determination is made as to whether or not the LEDs 58 of the therapy modules 52 are already warm. To determine if the LEDs 58 are warm, the microprocessor 166 counts the time since the end of the preceding therapy treatment. If the time from the preceding therapy treatment is more than a predetermined time, for example ten minutes, the microprocessor 166 determines that the LEDs 58 have had sufficient time to cool and are therefore no longer warm. It is important to determine if the LEDs 58 are warm or cool because the light intensity from the LEDs 58 is higher when they are cool than when they are hot (as shown and explained in conjunction with FIGS. 23 and 24).

If the determination at 392 is that the LEDs 58 are warm, the process flow 370 proceeds to 394 to where the treatment time is increased by the microprocessor 166 to compensate for the decreased intensity of the warm LEDs 58. At 394, the treatment time set at 390 is increased by an additional amount, for example 1.5 minutes. After the treatment time is increased at 394 the program proceeds to 396 where an aural indication is presented that the therapy modules 52 are delivering low-power treatment. If, on the other hand, the determination at 392 is that the LEDs 58 are cool, then the process flow 370 bypasses the step at 394 and goes directly the step at 396 to indicate aurally that the therapy modules 52 are delivering low-power treatment.

From 396 the process flow 370 proceeds to 378 where a determination is made whether a timing error has occurred. If the determination is affirmative, the process flow terminates at 380. If the determination at 378 is negative, the process flow proceeds to 374 to determine if a button has been pushed. So long as the determination at 374 is negative, indicating that neither button 70 or 72 has been pressed, the process flow proceeds to 376 where an affirmative determination occurs because low-power therapy treatment has commenced. The process flow advances from 376 to the determination at 398 where the microprocessor 166 determines if the therapy treatment is ended. The treatment is ended when an internal timer that was initially set at 390, and thereafter possibly increased at 394, has counted down to zero.

If the treatment has not ended as determined at 398, the process flow 370 enters a loop created by the negative determination at 398, the negative determination at 378, the negative determination at 374 and the affirmative determination at 376. This loop continues until a button is pressed as determined at 374, or until a clock timing error occurs as determined at 378, or until the treatment is ended as determined at 398.

When it is determined at 398 that the treatment is ended, the process flow advances to 400 where the microprocessor 166 deasserts the enable signal 288 which causes the power delivery switch 302 to cease delivering electrical power to the therapy modules 52 (FIG. 21). The end of the treatment is thereafter visually and audibly signaled at 402. The process flow moves to 378, where a check of timing errors is again made. If a timing error has occurred, all operations terminate at 380. If no timing error has occurred, the process flow advances to 374 and enters the sensing loop of 374, 376 and 378, to await the commencement of another treatment by a button push at 374.

If only the high-power button 70 is pressed instead of the low-power button 72, the process flow 370 exits the sensing loop 374, 376 and 378 with an affirmative determination at 374. A negative determination occurs at 382, followed by an affirmative determination at 404, both of which signify that only the high-power selector button 70 was pressed. In this instance, the process flow 370 advances from 404 to 405 where electrical power is supplied to the therapy modules 52 as a result of the power delivery switch 302 becoming conductive (FIG. 21). Following the initiation of power delivery at 405, the process flow 370 proceeds to 406 where the microprocessor 166 asserts a low level therapy control signal 246 to disconnect the auxiliary thermistor 109 from the main thermistor 108 by causing the switch 244 to become nonconductive (FIG. 18). As a result, the temperature reference signal 236 increases (FIG. 19B) and causes the therapy module 52 to deliver high-power therapy.

The process flow then advances to 408 where the high-power therapy is visually indicated. The process flow then advances to 410 where the internal timer of the microprocessor 166 is then set to the basic predetermined time established for high-power therapy. A determination of whether the LEDs 58 are warm occurs next at 412, by timing the interval since the last use of the therapy device 50, in the manner previously described. If it is determined that the LEDs 58 are warm, then the basic time established at 410 on the internal timer of the microprocessor 166 is increased at 414 by an amount to compensate for the decreased intensity of the warm LEDs 58, for example 1.7 minutes. Thereafter, the high-power therapy treatment is signaled aurally at 416. If the LEDs 58 are cool, as established by a negative determination at 412, the basic time for the high-power therapy treatment is not increased and the high-power therapy treatment is signaled aurally at 416.

From 416, the process flow proceeds to 378 to determine whether a timing error has occurred. An affirmative determination at 378 results in the termination of the treatment at 380. A negative determination at 378 advances the process flow to 374 where the determination is made if a button has been pushed. A negative determination at 374 places the process flow into the sensing loop waiting for either a button to be pressed as determined at 374, or the treatment to finish as determined at 398, or a timing error to occur as determined at 378.

When the high-power therapy treatment is ended, as determined at 398, the power to the therapy modules 52 is terminated, the LEDs 58 cease to emit light at 400 and the energy delivery LED 93 ceases to emit light. At 402 the end of high-power therapy treatment is signaled. The process flow moves to 378, where a check of timing errors is again made. If a timing error has occurred, all operations terminate at 380. If no timing error has occurred, the process flow advances to 374 and enters the sensing loop of 374, 376 and 378, to await the commencement of another treatment by a button push at 374.

At any time during a continuing therapy treatment, the user is able to stop the treatment by pressing both the high- and low-power buttons 70 and 72 at the same time. If both buttons 70 and 72 (FIG. 1) are pressed simultaneously, the determination at 374 is affirmative and the determinations at 382 and 404 are negative. Under these conditions, the process flow 370 advances to the determination at 418 where the microprocessor 166 determines that buttons 70 and 72 have been pressed simultaneously. An affirmative determination at 418 advances the process flow 372 to the determination at 420. A determination is made at 420 whether a treatment is in progress, as a result of the assertion of the enable signal 288 (FIG. 21). An affirmative determination at 420 results in the termination of electrical power to the therapy modules 52, causing the LEDs 58 and 93 to cease emitting light at 400. Thereafter the end of the treatment is signaled at 402. In this manner, simultaneously pressing both the low-power and high-power selector buttons ends any ongoing therapy treatment.

A negative determination at 418 would only occur if some error in the progress of the process flow 370 has occurred. A negative determination at 420 would occur if both the high-power and the low-power selector buttons were simultaneously pressed when no therapy treatment was being administered. If either determination at 418 or 420 is negative, and after signaling the end of treatment at 402, the process flow 370 advances to the determination at 378. The process flow moves to 378, where a check of timing errors is again made. If a timing error has occurred, all operations terminate at 380. If no timing error has occurred, the process flow advances to 374 and enters the sensing loop of 374, 376 and 378, to await the commencement of another treatment by a button push at 374.

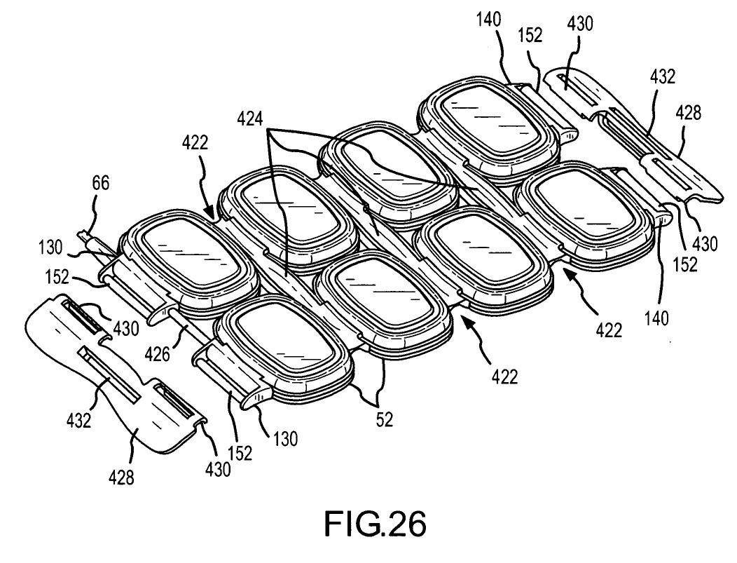

In some circumstances, the area of the tissue to be treated with the therapy device 50 is greater than the area which can be treated by the linear row of therapy modules 52 shown in FIGS. 1 and 2. In such circumstances, it is desirable to use a two-dimensional configuration of the therapy modules 52, as shown in FIG. 26, in place of the single linear row of therapy modules shown in FIG. 1. The two-dimensional configuration of therapy modules 52 permits the heat and light therapy to be delivered over a larger and more encompassing area of skin and tissue than can be treated by the single linear row of therapy modules 52.

The two-dimensional configuration of therapy modules 52 is formed by multiple single rows of the therapy modules. The two-dimensional configuration shown in FIG. 26 is formed by two rows of therapy modules 52. Each row of therapy modules 52 in the two-dimensional configuration has the same basic characteristics previously described. Cross couplers 422 link the laterally adjacent therapy modules 52 in the multiple rows. The cross couplers 422 are similar to the couplers 54 (FIG. 1) in the single row of therapy modules 52, except that they include cross straps 424 which extend and connect to the couplers of laterally adjacent therapy modules 52. The cross straps 424 of the cross couplers 422 extend between the laterally spaced rows of therapy modules 52 to separate the therapy modules at uniform lateral distances with respect to one another. The cross straps 424 have a resilient flexible characteristic which allows the therapy modules to flex in a lateral sense with respect to one another. The cross straps 424 are preferably made from the same material as the couplers 54 (FIG. 1). Each cross coupler 422 is overmolded and surrounds a single flexible circuit which extends longitudinally along each row of therapy modules 52 in the two-dimensional configuration in the same manner as has previously been described in conjunction with the single linear row of therapy modules (FIG. 1).

Each row of therapy modules 52 in the two-dimensional configuration includes a cable end terminal coupler 130 and a row end terminal coupler 140. The cable 66 from the control module 64 (FIG. 1) connects to the cable end terminal coupler 130 on one of the lateral end rows of therapy modules. A short flexible electrical extension cable 426 extends between the cable end terminal coupler 130 to which the cable 66 is attached and the adjacent cable end terminal coupler 130 on the laterally adjacent row of therapy modules. In this manner, the electrical power and the therapy control signal are communicated between all the rows in the two-dimensional configuration. As an alternative to using the extension cable 426, conductors may extend through the cross straps 424 to electrically connect corresponding traces of the flexible circuits which extend along each row of the therapy modules 52, thereby supplying the electrical power and the therapy control signal to all of the rows of therapy modules in the two-dimensional configuration.

Each of the terminal couplers 130 and 140 includes the connection shaft 152. A single somewhat-flexible strap connector 428 includes multiple hook portions 430 which align with and connect to the connection shaft 152 in the same manner that the single hook portion 150 of the hook clasp 144 connects to the connection shaft 52 (FIGS. 13 and 14). The strap connector 428 also includes a slot 432 through which a strap can be inserted and connected to the strap connectors 428 in the same manner as the strap is connected to the single hook clasps 144 (FIGS. 1 and 13-15).

The multi-row, two-dimensional configuration of therapy modules 52 is controlled and powered by the same control module 64 which is used to control and power the single linear row of control modules. However, the number of rows of therapy modules in the two-dimensional configuration must not be so large as to exceed the capacity of the electronic components within the control module 64.

Numerous improvements and advantageous features of the therapy device 50 have been discussed above. By individually controlling the heat and light output energy from each therapy module 52 based on a sensed temperature, each therapy module 52 is able to deliver the maximum therapeutic effect at each individual location treated by each therapy module. Good thermal contact with the skin of the user is achieved. Internal timing errors that may lead to the prolonged treatment are avoided by monitoring the frequency of the internal clock of the microprocessor. A reduction in the light intensity output from warm LEDs 58 is compensated for by adjusting the basic treatment time. The amount of light energy applied can be more accurately determined and predicted. Allowing the user to choose either a high-power therapy treatment or a low-power therapy treatment, and adjusting the treatment time accordingly, allows the user to express his or her treatment preferences without compromising the amount of therapy delivered.

The therapy modules 52 are flexibly and adaptably coupled to permit the therapy modules to better contact and follow the contour of the user's anatomy, thereby allowing the heat and light therapy treatment to be applied effectively over a variety of different locations on the human body. Relatively large areas of tissue may be treated simultaneously by the use of the relatively larger two-dimensional configuration of therapy modules. The connection straps hold the therapy modules in contact with the user's skin and permit the therapy modules to be quickly and conveniently positioned and attached for use, as well as permitting the therapy modules to be easily disconnected and removed at the conclusion of the therapy treatment.

Other improvements and advantages are either discussed above or will be more apparent upon fully comprehending the significant aspects of the present invention. The presently preferred embodiments of the invention have been described above with a degree of particularity. The description is of preferred examples for implementing the invention, and is not necessarily intended to limit the scope of the invention. The scope of the invention is defined by the following claims.

The invention claimed:

1. A therapy device for generating heat and light and applying the generated heat and light to skin of a user, comprising:
a plurality of therapy modules, each therapy module including an array of a plurality of light emitting devices which generate heat when emitting light, each therapy module having a housing with a window through which the light and heat generated by the light emitting devices passes, each therapy module further including electronic circuitry located within the housing with which to apply electrical energy to the light emitting devices to cause them to generate the light and heat;
at least one flexible coupler connecting each adjoining pair of therapy modules into a single configuration of the plurality of therapy modules, each flexible coupler including electrical conductors for conducting electrical power between the electronic circuitry located within the housings of the adjoining pairs of therapy modules; and
a control module connected by a cable to one of the plurality of therapy modules, the control module including circuit components which supply electrical power through the cable to the electronic circuitry located in the housing of the one therapy module, the conductors of the flexible couplers distributing the electrical power from the one therapy module to the other therapy modules in the single configuration.

2. A therapy device as defined in claim 1, wherein:
the electronic circuitry located within the housing of each therapy module includes a temperature sensor in thermally conductive contact with the skin of the user; and
the electronic circuitry of each therapy module modulates the electrical energy applied to the light emitting devices of that therapy module to control the temperature of the skin adjacent to that therapy module by controlling the light and heat emitted from the light emitting devices within each therapy module.

3. A therapy device as defined in claim 2, wherein:
the window includes a protrusion extending outward from the window to physically contact the skin of the user;
the window includes a stud extending into the housing of the therapy module on the opposite side of the protrusion;
the protrusion thermally contacts the temperature sensor; and
the protrusion and the stud establish a thermally conductive path directly from the skin of the user to the temperature sensor.

4. A therapy device as defined in claim 1, wherein:
the circuit components of the control module include a controller for timing the duration of electrical power supplied through the cable to the single configuration of therapy modules, the controller also initiating the supply of electrical power at the commencement of a therapy treatment and terminating the supply of electrical power at the end of the therapy treatment.

5. A therapy device as defined in claim 4, wherein:
the circuit components of the control module deliver a clock signal at a predetermined frequency which is used for timing the duration of the therapy treatment; and
the circuit components of the control module monitor the clock signal for deviations from the predetermined frequency and terminate the supply of electrical power upon detecting that the frequency of the clock signal has deviated by a predetermined amount from the predetermined frequency.

6. A therapy device as defined in claim 4, wherein:
the controller measures the time between the termination of a preceding therapy treatment and the commencement of a subsequent therapy treatment and adds a predetermined amount of time to the duration of electrical power supplied through the cable to the single configuration of therapy modules when the measured time between the termination of the preceding therapy treatment and the commencement of the subsequent therapy of treatment indicates that the light emitting devices will emit light of reduced intensity due to residual temperature of the light emitting devices resulting from the preceding therapy treatment.

7. A therapy device as defined in claim 4, wherein:
the circuit components of the control module deliver a clock signal at a predetermined frequency which is used for timing the duration of the therapy treatment;
the circuit components of the control module include low-power and high-power control switches which are selectively activated to create a relatively longer time duration for a low-power therapy treatment and a relatively shorter time duration for a high-power therapy treatment, respectively;
the circuit components of the control module assert a therapy control signal indicating the selected one of the low-power or high-power therapy treatments on a conductor of the cable to the electronic circuitry of the one therapy module;
the flexible coupler further includes a conductor for conducting the therapy control signal between the electronic circuitry located within the housings of the adjoining pairs of therapy modules;
the electronic circuitry of each therapy module modulates the electrical energy applied to the light emitting devices to establish a relatively greater amount of light and heat emitted from the light emitting devices within a specific time upon the therapy control signal indicating the selection of the high-power therapy treatment; and
the electronic circuitry of each therapy module modulates the electrical energy applied to the light emitting devices to establish a relatively lesser amount of light and heat emitted from the light emitting devices within the specific time upon the therapy control signal indicating the selection of the low-power therapy treatment.

8. A therapy device as defined in claim 1, wherein:
the conductors within each flexible coupler comprise traces on a flexible circuit, the flexible circuit having a substantially flat continuous flexible insulating substrate upon which traces are formed as the electrical conductors, and
each flexible coupler comprises flexible plastic material which is molded over and surrounds the flexible circuit, the flexible plastic material mechanically connecting to the housings of the adjoining therapy modules.

9. A therapy device as defined in claim 8, wherein:
the flexible circuit extends substantially through the housing of each therapy module;
the flexible plastic material of each flexible coupler terminates within each housing of each therapy module to expose a portion of the flexible circuit within each housing of each therapy module; and
the electronic circuitry of each therapy module is electrically connected to the traces on the exposed portion of the flexible circuit within each housing of each therapy module.

10. A therapy device as defined in claim 9, wherein:
the electronic circuitry of each therapy module is attached to a circuit board located within the interior of the housing of each therapy module;
the circuit board is oriented within the housing of each therapy module to extend generally parallel to the window;
the light emitting devices are attached to the circuit board between the circuit board and the window;
a substantial majority of the electronic circuitry of each therapy module is attached on the opposite side of the circuit board from the window; and
the exposed portion of the flexible circuit is electrically connected to the circuit board on the opposite side of the circuit board from the window.

11. A therapy device as defined in claim 10, wherein:
the electronic circuitry located within the housing of each therapy module includes a first temperature sensor in thermally conductive contact with the skin of the user;
the electronic circuitry located within the housing of each therapy module also includes a second temperature sensor connected to the circuit board on the opposite side from the light emitting devices; and
the first temperature sensor is connected to the circuit board on the same side as the light emitting devices.

12. A therapy device as defined in claim 11, wherein:
the electronic circuitry of each therapy module modulates the electrical energy applied to the light emitting devices of that therapy module to control the temperature of the skin adjacent to that therapy module by controlling the light and heat emitted from the light emitting devices in response to the temperatures sensed by either one or both the first and second temperature sensors.

13. A therapy device as defined in claim 10, wherein:
the electronic circuitry attached on the opposite side of the circuit board from the light emitting devices includes an energy delivery indicating light emitting device which is energized to deliver light when the light emitting devices which deliver the heat and light energy to the skin of the user are energized.

14. A therapy device as defined in claim 13, wherein:
the electronic circuitry of each therapy module modulates the electrical energy applied to the light emitting devices to control the light and heat emitted from the light emitting devices within each therapy module; and
the electronic circuitry of each therapy module modulates the electrical energy applied to the energy delivery indicating light emitting device to indicate the modulation of the electrical energy applied to the light emitting devices which deliver light and heat to the skin of the user, the modulation of the electrical energy applied to the energy delivery indicating light emitting device creating a modulation in intensity of light from the energy delivery indicating light emitting device.

15. A therapy device as defined in claim 1, wherein:
the plurality of therapy modules in the configuration form a linear row;
the therapy modules at the end of the linear row include terminal couplers connected on the opposite side of each therapy module from the flexible couplers which connect the therapy modules in the row; and
the terminal couplers include connectors by which to attach a strap extending between both terminal couplers, the strap for holding the linear row of therapy modules in contact with the skin of the user.

16. A therapy device as defined in claim 15, wherein:
each end of the strap is directly connected to each terminal coupler.

17. A therapy device as defined in claim 16, wherein:
each end of the strap includes a connection device for mechanically connecting to each terminal coupler.

18. A therapy device as defined in claim 15, wherein:
the cable from the control module is connected to one terminal coupler; and
the electrical conductors of the cable are electrically connected to electrical conductors and connected to the electronic circuitry of the therapy module.

19. A therapy device as defined in claim 18, wherein:
the conductors within each flexible coupler comprise traces on a flexible circuit, the flexible circuit having a substantially flat continuous flexible insulating substrate upon which traces are formed as the electrical conductors;
the flexible circuit extends substantially through the housing of each therapy module in the linear row and into each terminal coupler;
each flexible coupler and each terminal coupler comprises flexible plastic material which is molded over and surrounds the flexible circuit, the flexible plastic material mechanically connecting to the housings of the therapy modules.

20. A therapy device as defined in claim 1, wherein:
the plurality of therapy modules form a two-dimensional configuration.

21. A therapy device as defined in claim 20, wherein:
the two-dimensional configuration of therapy modules is formed by a plurality of laterally adjacent linearly connected rows of therapy modules.

22. A therapy device as defined in claim 1, wherein:
the control module includes a body within which the circuit components of the control module are located; and
the body includes an attachment clip for mechanically connecting the control module to an object worn by the user.

23. A method for generating heat and light and applying the generated heat and light to the skin of a user, comprising:
organizing a plurality of light emitting devices in an array;
generating heat and light by supplying electrical energy to each light emitting device in the array;
positioning a plurality of separate arrays to deliver heat and light to substantially adjoining but separate areas of the user's skin; and
separately controlling the electrical energy applied to the light emitting devices of each array to control the temperature of the skin at each separate area independently of the temperature of the skin at the other separate areas.

24. A method as defined in claim 22, further comprising:
flexibly coupling together the plurality of separate arrays;
applying electrical energy to the light emitting devices of each array through at least one flexible coupling to each array;
conducting electrical energy between the electronic circuitry of the adjoining pairs of therapy modules; and
supplying the electrical energy through a cable to one array and distributing the electrical energy from the one array through flexible couplings to the other arrays of the plurality.

25. A method as defined in claim 24, further comprising:
flexibly coupling together the plurality of arrays with a flexible circuit, the flexible circuit having a substantially flat continuous flexible insulating substrate upon which traces are formed as the electrical conductors by which to deliver the electrical energy to the plurality of arrays.

26. A method as defined in claim 25, further comprising:
molding flexible plastic material over and surrounding the flexible circuit between individual arrays.

27. A method as defined in claim 23, further comprising:
sensing the temperature of the skin of the user through direct thermal contact.

28. A method as defined in claim 23, further comprising:
timing the duration of electrical energy supplied to the arrays to establish a therapy treatment duration.

29. A method as defined in claim 28, further comprising:
delivering a clock signal at a predetermined frequency by which to time the duration of the therapy treatment;
monitoring the clock signal for deviations from the predetermined frequency; and
terminating the supply of electrical energy upon detecting that the frequency of the clock signal has deviated by a predetermined amount from the predetermined frequency.

30. A method as defined in claim 28, further comprising:
measuring the time between the termination of a preceding therapy treatment and the commencement of a subsequent therapy treatment; and
adding a predetermined amount of time to the duration of the therapy treatment when the measured time between the termination of the preceding therapy treatment and the commencement of the subsequent therapy treatment indicates that the light emitting devices will emit light of reduced intensity due to the residual temperature of the light emitting devices resulting from use during the preceding therapy treatment.

31. A method as defined in claim 28, further comprising:
delivering a clock signal at a predetermined frequency by which to time the duration of the therapy treatment;
selecting one of either a low-power therapy treatment having a relatively longer time duration or a high-power therapy treatment having a relatively shorter time duration;
signaling the selected one of the low-power or high-power therapy treatment to each array of light emitting devices; and
separately controlling the amount of electrical energy applied to the light emitting devices of each array to increase the amount of light and heat emitted from the light emitting devices in a specific time upon selecting the high-power therapy treatment and to decrease the amount of light and heat emitted from the light emitting devices in the specific time upon selecting the low-power therapy treatment.

32. A method as defined in claim 23, further comprising:
thermally conducting the temperature of the user's skin at each of the separate areas to a first temperature sensor associated with each array;
sensing the temperature generally surrounding the array with a second temperature sensor associated with each array; and
separately controlling the electrical energy applied to the light emitting devices of each array in response to the temperatures sensed by either one or both of the first and second temperature sensors.

33. A method as defined in claim 24, further comprising:
orienting the plurality of arrays in a linear row;
connecting a strap to an end of the linear row; and
holding the linear row on the user with a strap.

34. A method as defined in claim 23, further comprising:
separately controlling the amount of electrical energy applied to the light emitting devices of each array to control the amount of light and heat delivered at each separate area; and
visually indicating with an energy delivery indicating light emitting device which is separate from the light emitted from the array of light emitting devices that the light emitting devices are energized to deliver light and heat energy to the skin of the user.

35. A method as defined in claim 34, further comprising:
modulating the electrical energy applied to the light emitting devices to control the light and heat emitted from the light emitting devices within each therapy module;
modulating the electrical energy applied to the energy delivery indicating light emitting device to indicate the modulation of the electrical energy applied to the light emitting devices which deliver light and heat to the skin of the user; and
modulating the intensity of light from the energy delivery indicating light emitting device in relation to the modulation of the electrical energy applied to the energy delivery indicating light emitting device.

36. A method as defined in claim 23, further comprising:
orienting the plurality of arrays into a two-dimensional configuration formed from a plurality of laterally adjacent rows of arrays.
